(19) United States
(12) Patent Application Publication
(10) Pub. No.: US 2006/0046273 A1
(43) Pub. Date: Mar. 2, 2006

(54) Homogeneous enzyme immunoassay for oral fluid

(75) Inventors: Cheng-I Lin, Cupertino, CA (US); Marie Lin, Cupertino, CA (US); Tom Chia, Fountain Valley, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Lin-Zhi International Inc., Sunnyvale, CA (US)

(21) Appl. No.: 10/927,823

(22) Filed: Aug. 27, 2004

(51) Int. Cl. G01N 33/542; G01N 33/53

(52) U.S. Cl. 435/7.9

(57) ABSTRACT

The present invention discloses homogeneous enzyme immunoassay systems, methods and kits useful for the qualitatively and quantitatively determination of analytes in oral fluid samples. The system involves a competitive enzyme immunoassay employing a conjugate comprising glucose-6-phosphate dehydrogenase (G6PDH) and an analyte. The methods and kits are particularly useful in the detection of recent drug use and for fast determination of analytes using auto-analyzers.

### FIELD OF THE INVENTION

The present invention relates to the field of immunoassays. The invention provides compositions and methods for determining the amount of an analyte in an oral fluid specimen suspected of containing the analyte. In particular, the immunoassays compositions of this invention comprise a glucose-6-phosphate dehydrogenase (G6PDH)-analyte conjugate, an antibody reactive to the analyte, an oral fluid sample suspected of containing the analyte, an enzyme substrate, and a co-enzyme for G6PDH. The invention also relates to kits useful for performing measurements of analytes in oral fluid specimen using homogeneous immunoassays.

### BACKGROUND OF THE INVENTION

There is a continuing interest in developing new, simpler more rapid and more sensitive techniques to measure the presence of an analyte in a sample suspected of containing an analyte. In particular, the measurement of trace amounts of analytes, particularly chemical substances, has become essential for many health care applications in pharmaceutical studies, therapeutic drug monitoring, and for drug abuse detection. There are a number of occasions on which a sample, particularly a sample of a biological fluid obtained from an individual, is to be screened for the presence of one or more analytes. For instance, samples of blood, urine, or other biological fluids from applicants for certain permits or licenses may be checked for the presence of alcohol or illicit drugs. Samples from a driver may be checked for such substances after an accident, or in applying for commercial permits or licenses or their renewals. Samples of individuals undergoing a drug treatment program may be screened for the presence of drugs. Samples from athletes may be screened to determine the presence of banned substances such as drugs, steroids, or other performance-enhancing substances.

Such screening may be done for substances other than illicit drugs or the like. For instance, patients admitted to a hospital may need to be checked for both licit and illicit drugs, including tranquilizers, and the like, so that appropriate treatment may be given or precautions taken. Such patients may be unconscious or suffering from trauma and may be unable to volunteer, or may be unwilling to provide, information about ingestion of certain substances. Checking of employees, workers or other persons at a certain location may need to be conducted to ascertain whether the individual has been exposed to a plurality of chemicals used in or around the workplace, or released into the environment.

In all the above cases, the screening is typically done to determine whether the substances in question are present in the bodies (that is, in samples of biological fluids) of the individuals in question. Typically the screening is to be conducted not only to determine whether detectable amounts of the substances in question are present in the sample, but whether a particular substance is present in an amount greater than a predetermined level. Such a level is also known as a “cutoff level.” These levels may be set by an organizational rule, e.g. an employer's rule, or by a law, for example, a maximum level of blood alcohol for one driving a vehicle, or a maximum amount of a steroid or other performance-enhancing substance for one to compete in an athletic event.

A wide variety of patents and publications disclose different immunoassay techniques (see, for example, U.S. Pat. Nos. 3,646,346 and 4,062,733, employing radioactive label; U.S. Pat. Nos. 3,654,090, 3,791,932 and 3,817,837, employing enzyme labels; U.S. Pat. No. 3,996,345, employing fluoresescer-quencher labels; U.S. Pat. No. 4,067,959, employing fluorescer or enzyme labels; and U.S. Pat. No. 4,160,645, employing non-enzymatic catalyst labels). Typically, these immunoassays employ an antibody which will specifically recognize the analyte to be tested for and a signal-producing system that produces a detectable change in signal in response to the binding of the antibody to the analyte.

Specifically, U.S. Pat. No. 3,817,837 provides a typical method for conducting assays for screening for the presence of individual analytes in a sample, using an enzyme amplification assay, and describes procedures for detecting the presence of a number of different types of chemical substances, including licit and illicit drugs. The procedure involves a competitive binding assay of the drug, either per se or in a form that contains a linking group that can bind to the enzyme used in the procedure. Inhibition of enzymatic activity is utilized to determine the presence and quantity of the chemical substance present in the sample. The method is frequently referred as the Enzyme Multiplied Immunoassay Technology (EMIT). This patent is hereby incorporated herein, in its entirety.

U.S. patent application Ser. No. 10/163,018 (Publication No. US-2003-0224373-A1), hereby incorporated in its entirety, describes a homogenous enzyme immunoassay for the simultaneous detection of multiple analytes. The homogeneous enzyme immunoassay relies on the formation of an enzyme-analyte conjugate to modulate a signal obtained from the enzyme. Further modulation of the enzymatic activity of the enzyme-analyte conjugate, i.e., a different level of signal produced by the enzyme within the conjugate, was achieved by binding of an antibody to the analyte of the enzyme-analyte conjugate. This result may be explained by the antibody binding to the analyte and causing a steric inhibition of the enzyme or an allosteric modification of the enzyme activity. The presence of an analyte in a test sample may reduce the amount of antibody bound to the enzyme-analyte conjugate and thereby cause a change in enzymatic activity of the enzyme-analyte conjugate. Thus, by measuring the signal generated by the enzyme, one can correlate the level of the signal to an amount of analyte in the test sample.

U.S. Pat. Nos. 6,033,890, 6,090,567, and 6,455,288 disclose conjugates and methods for immunoassays of analytes using mutant glucose-6-phoshate dehydrogenase (G6PDH). In particular, the invention of U.S. Pat. No. 6,033,890 relates to the use of conjugates of an analyte or analyte analog and a mutant NAD⁺ dependant G6PDH differing from any precursor G6PDH by deletion, substitution, or insertion, or any combination thereof of at least one amino acid per subunit. However, none of the U.S. Pat. Nos. 6,033,890, 6,090,567, and 6,455,288, teach using the invention for analyzing oral fluid samples. Hereby, the disclosures U.S. Pat. Nos. 6,033,890, 6,090,567, and 6,455,288 are incorporated by reference in their entirety.

Because of higher analyte concentration in urine, blood, serum or plasma samples, these bodily fluids represented the samples of choice for analyte measurements. However, privacy concerns of individuals whose samples will be analyzed coupled with the desire or need to visually control collection of the test sample and considering health issues (HIV, hepatitis, etc.) involved in collecting blood, serum or plasma samples often make the collection of these samples impractical. Thus, it would be much more desirable to collect oral fluid samples instead of urine, blood, serum or plasma samples.

Oral fluid has recently been widely used as a specimen in pharmaceutical studies, therapeutic drug monitoring, and for detection of drug abuse. The currently available oral fluid testing methods include conventional ELISA and on-site dip-stick testing, which are time consuming, labor intensive, costly, and of low precision.

Despite the widespread use of immunoassays, there are still difficulties in the measurement of analytes derived from particular sample types, notably oral fluids, in which the analyte may be present at very low concentrations. Thus, while the techniques described in U.S. Pat. Nos. 3,817,837, 6,033,890, 6,090,567, and 6,455,288 and U.S. patent application Ser. No. 10/163,018 substantially improved the efficiency of testing various analytes, they do not resolve the problem of measuring the presence of an analyte in a fluid sample of low analyte concentration, such as an oral fluid sample. The main difficulty of applying the homogeneous enzyme immunoassay, described in U.S. patent application Ser. No. 10/163,018, to oral fluid is due to the low analyte concentration in the oral fluid specimen.

For example, guidelines provided by the Substance Abuse and Mental Health Services Administration (SAMHSA; Federal Register, 2004; 69(71), 19673-19719) show that the detection limit, i.e., the recommended cutoff levels for various analytes in oral fluid samples are much lower than those for urine samples:

| Drug Category | Urine | Oral Fluid (OF) | Treated OF | |
|---|---|---|---|---|
| Amphetamines | 1000 ng/mL | 50 ng/ml | 12.5-25 | ng/ml |
| Cocaine metabolite | 150 ng/mL | 20 ng/ml | 5-10 | ng/ml |
| Methamphetamine | 500 ng/ml | 50 ng/ml | 12.5-25 | ng/ml |
| Ecstasy (MDMA) | 300 ng/ml | 50 ng/ml | 12.5-25 | ng/ml |
| Methadone | 300 ng/ml | 40 ng/ml | 10--20 | ng/ml |
| Opiate | 2000 ng/mL | 40 ng/ml | 10-20 | ng/ml |
| Phencyclidine | 25 ng/mL | 10 ng/ml | 2.5-5 | ng/ml |
| THC | 50 ng/mL | 4 ng/ml | 1-2 | ng/ml |

However, the low cutoff concentrations of analyte in oral fluid make it impossible to simply apply methods used for urine testing to oral fluid specimen. Measuring the already low analyte concentration in oral fluid samples may be even more challenging due to oral fluid collection procedures during which oral fluid samples are preserved by the addition of certain buffers. Usually this preservation process dilutes the analyte concentration an additional 2 to 4 fold decreasing the analyte concentration further lower (see treated OF, above). Thus, for example, detection of 1-2 ng/ml of THC must be achieved according to the federal guidelines.

Thus, in developing an assay for detecting analytes in oral fluid samples, there are many considerations, not the least of which is sensitivity. Another consideration is interference by materials present in oral fluid samples or interference due to viscosity of the oral fluid sample. None of the above patents presented a solution to these problems.

Thus, it is an objective of this invention to provide an assay system, a method and a kit that can be used to quickly and reliably determine the amount of an analyte in an oral fluid specimen with certain specific, relevant cutoffs. In particular, it is an objective to increase the sensitivity of currently available assays to meet the federal standards for the detection and determination of illicit drugs in oral fluid samples.

The present invention overcomes the current obstacles and provides homogeneous enzyme immunoassay systems, methods and kits useful for the qualitatively and quantitatively determination of low concentration analytes in oral fluid specimen. The system involves a competitive enzyme immunoassay employing a conjugate comprising glucose-6-phosphate dehydrogenase (G6PDH) and an analyte. The methods and kits are particularly useful in the detection of recent drug use and for fast determination of analytes using auto-analyzers.

### SUMMARY OF THE INVENTION

In its broadest application, the present invention can be used to measure any analyte in any sample. In its narrowest application, the present application provides a quantitative homogeneous enzyme immunoassay specific for oral fluid samples based upon the specific reversible inhibition of a glucose-6-phosphate dehydrogenase (G6PDH)-analyte conjugate/antibody complex by free analyte in the oral fluid sample.

This invention comprises a homogeneous enzyme immunoassay system for determining the amount of an analyte in an oral fluid sample or oral fluid specimen. The system involves a homogeneous enzyme immunoassay, which has a dynamic range of 0-100 ng/ml and produces an absorbance signal within the dynamic range from 0 to greater than 100 milli-absorbant units with a coefficient of variation of less than 10%. The system further comprises an aqueous medium comprising (a) an enzyme-analyte conjugate comprising glucose-6-phosphate dehydrogenase (G6PDH) covalently linked to an analyte; (b) an antibody reactive to the analyte; (c) an oral fluid sample suspected of containing the analyte; (d) an enzyme substrate for G6PDH; and (e) a co-enzyme for G6PDH. It is further provided that (i) the G6PDH has a starting specific activity of at least 800 units/mg and the enzyme-analyte conjugate is deactivated from about 30% to about 65% due to covalent linkage of the G6PDH to the analyte and (ii) wherein the deactivated enzyme-analyte conjugate is further inhibited from about 40% to about 85% due to binding of the antibody to the analyte of the enzyme-analyte conjugate.

In other embodiments of this invention, the oral fluid sample is buffered to a pH range from between 7.2 and 8.3. In another embodiment, the oral fluid sample is filtered or centrifuged.

In one embodiment of the invention, the analyte is selected from the group consisting of licit drugs, illicit drugs and analogs, derivatives and metabolites thereof. In another embodiment the analyte is selected from the group consisting of opium, opioid analgesics, amphetamines, cocaine, methadone, methadone metabolite, MDMA, PCP, propoxyphene, benzodiazepines, barbiturates, THC, alcohol and analogs, metabolites, and derivatives thereof.

In one embodiment of this invention, the G6PDH is obtained from a natural source. In another embodiment, G6PDH is a recombinant enzyme.

In one embodiment of this invention, the oral fluid sample is between about 20 μl and about 50 μl in volume.

The invention also provides methods for determining the amount of an analyte in an oral fluid sample. The method involves a homogeneous enzyme immunoassay, which has a dynamic range of 0-100 ng/ml and produces an absorbance signal within the dynamic range from 0 to greater than 100 milli-absorbant units with a coefficient of variation of less than 10%. The method further comprises the steps of (I) combining in an aqueous medium (a) an enzyme-analyte conjugate comprising glucose-6-phosphate dehydrogenase (G6PDH) covalently linked to an analyte; (b) an antibody reactive to the analyte; (c) an oral fluid sample suspected of containing the analyte; (d) an enzyme substrate for G6PDH; and (e) a co-enzyme for G6PDH and (II) detecting a change in enzymatic activity of the enzyme-analyte conjugate due to competitive binding of the antibody to the analyte of the enzyme-analyte conjugate and the analyte in the oral fluid sample. It is further provided that (i) the G6PDH has a starting specific activity of at least 800 units/mg and the enzyme-analyte conjugate is deactivated from about 30% to about 65% due to covalent linkage of the G6PDH to the analyte and (ii) wherein the deactivated enzyme-analyte conjugate is further inhibited from about 40% to about 85% due to binding of the antibody to the analyte of the enzyme-analyte conjugate and (iii) wherein the change in enzymatic activity is related to the amount of the analyte in the oral fluid sample.

The methods of this invention embrace the specifics outlined above for the immunoassay system.

The invention further provides kits for determining the amount of an analyte in an oral fluid sample suspected of containing an analyte using the methods of the present invention, the kit comprising in a packaged combination, one or more reagent compositions comprising (a) an enzyme-analyte conjugate comprising glucose-6-phosphate dehydrogenase (G6PDH) covalently linked to an analyte; (b) an antibody reactive to the analyte; (c) an enzyme substrate for G6PDH; and (d) a co-enzyme for G6PDH. In another embodiment, the kit also comprises an oral fluid calibrator.

### BRIEF DESCRIPTION OF THE DRAWINGS

FIG. 1 depicts a graph showing a calibration curve obtained by determining the amount of amphetamine using the oral fluid (OF) homogeneous enzyme immunoassay (EIA). The graph was prepared by plotting the results obtained in Example 7, in which samples containing amphetamine were assayed according to the present invention. The concentration of amphetamine is plotted on the X-axis and absorbance at 340 nm is plotted on the Y-axis.

FIG. 2 depicts a graph showing a calibration curve obtained by determining the amount of phencyclidine (PCP) using the oral fluid (OF) homogeneous enzyme immunoassay (EIA). The graph was prepared by plotting the results obtained in Example 8, in which samples containing phencyclidine were assayed according to the present invention. The concentration of phencyclidine is plotted on the X-axis and absorbance at 340 nm is plotted on the Y-axis.

FIG. 3 depicts a graph showing a calibration curve obtained by determining the amount of opiate using the oral fluid (OF) homogeneous enzyme immunoassay (EIA). The graph was prepared by plotting the results obtained in Example 10, in which samples containing opiate were assayed according to the present invention. The concentration of opiate is plotted on the X-axis and absorbance at 340 nm is plotted on the Y-axis.

FIG. 4 depicts a graph showing a calibration curve obtained by determining the amount of cocaine metabolite using the oral fluid homogeneous enzyme immunoassay. The graph was prepared by plotting the results obtained in Example 12, in which samples containing cocaine metabolite were assayed according to the present invention. The concentration of cocaine metabolite is plotted on the X-axis and absorbance at 340 nm is plotted on the Y-axis.

FIG. 5 depicts a graph showing a calibration curve obtained by determining the amount of Ecstacy (MDMA) using the oral fluid (OF) homogeneous enzyme immunoassay (EIA). The graph was prepared by plotting the results obtained in Example 13, in which samples containing Ecstacy (MDMA) were assayed according to the present invention. The concentration of Ecstacy (MDMA) is plotted on the X-axis and absorbance at 340 nm is plotted on the Y-axis.

FIG. 6 depicts a graph showing a calibration curve obtained by determining the amount of methadone metabolite (EDDP) using the oral fluid (OF) homogeneous enzyme immunoassay (EIA). The graph was prepared by plotting the results obtained in Example 14, in which samples containing methadone metabolite (EDDP) were assayed according to the present invention. The concentration of methadone metabolite (EDDP) is plotted on the X-axis and absorbance at 340 nm is plotted on the Y-axis.

### Definitions

As used herein:

“About” refers to a range of values of plus or minus 10% of a specified value. For example, the phrase “about 200” includes plus or minus 10% of 200, or from 180 to 220.

“Absorbance” or “absorbance signal” means a signal measured in a spectrophotometer or similar device. The signal is given as ‘absorbant unit’ or as ‘milli-absorbant unit.’

“Analyte” means a substance, compound or composition whose presence or concentration in a sample or specimen is to be determined. Within the context of the current patent, “analyte” may be used in substitution for “analyte and/or hapten” for fluidity and verbiage redundancy reduction. It is also equivalent to the word “ligand” as used in U.S. Pat. No. 3,817,837. More specifically, the term when used in the context of an enzyme-analyte conjugate, may include a drug, a metabolite of the drug or a representative epitope of the drug. Analytes may be monoepitopic or polyepitopic.

“Antibody” refers to a protein functionally defined as a binding protein (a molecule able to bind to a specific epitope on an antigen) and structurally defined as comprising an amino acid sequence that is recognized by one of skill as being derived from the framework region of an immunoglobulin encoding gene. It includes whole antibody, functional fragments, modifications or derivatives of the antibody. It can also be a genetically manipulated product, or chimeric antibody, such as a humanized antibody. Antibodies can be a polyclonal mixture or monoclonal. Antibodies can be intact immunoglobulins derived from natural sources or from recombinant sources and can be immunoreactive portions of intact immunoglobulins. Antibodies may exist in a variety of forms including, for example, Fv, Fab, and F(ab)₂, as well as in single chains. Single-chain antibodies, in which genes for a heavy chain and a light chain are combined into a single coding sequence, may also be used.

“Antibody reactive to analyte” means that the antibody has an area on its surface or in a cavity which specifically binds to a particular analyte, i.e., it has a binding affinity (usually expressed as Ka) for the analyte.

“Biological fluid” refers to a fluid from a host and includes whole blood, serum, plasma, urine, tears, mucus ascites fluid, oral fluid, semen, stool, sputum, cerebrospinal fluid and fetal fluid.

“Biological sample” refers to any sample obtained from a living or dead organism. Examples of biological samples include biological fluids specimens.

“Calibration material” refers to any standard or reference material containing a known amount of the analyte to be measured.

“Coefficient of variation” or “CV” or “% CV” means standard variation divided by average.

“Co-enzyme” or “co-factor” means a substrate necessary for an enzyme to catalyze a reaction.

“Competitive assay” means an assay in which an antibody bound to an enzyme-analyte conjugate competes for binding with an analyte present in a test sample. The two analyte-species, the “analyte” in the test sample and the enzyme-analyte conjugate may be added to the antibody or receptor solution simultaneously or sequentially.

“Conjugation” is any process wherein two subunits are linked together to form a conjugate, in particular and within the context of the present invention, an enzyme-analyte conjugate.

“Covalently linked” means two molecules linked to each other.

“Cutoff level”, “concentration of the cutoff” or “cutoff concentration” refer to a concentration of a given analyte to be tested for, at or above a predetermined concentration. A cutoff level tends to be a concentration established by a rule or standard of a government agency or of a governing body, for example, a governing body of a sport. Guidelines for cutoff levels are also provided by The National Institute of Drugs of Abuse (NIDA) and The Substance Abuse and Mental Health Services Administration (SAMHSA).

“Deactivated” or “Deactivation” refers to the capability of an analyte upon binding, covalent linkage or conjugation to an enzyme, to lower or decrease the activity of the enzyme. In particular, in this invention, the activity of the enzyme glucose-6-phosphate dehydrogenase (G6PDH) is deactivated upon conjugation of the analyte to G6PDH.

“Drug” refers to a substance, compound or composition, which includes both licit and illicit drugs, substances used for medicinal or pharmaceutical effects as well as substances used for producing narcotic or other addictive properties. As used herein, the term “drug” may also refer to chemical substances to be determined that are not strictly considered drugs, but that may be ingested by athletes for performance-enhancing effects (including nutritional substances), and whose presence is thus sought to be determined in screening samples from athletes. Such substances include, for instance, amino acids, steroids, and hormones, etc.

“Dynamic range” means the range of analyte concentration to be measured.

“Enzyme-analyte conjugate” refers to a covalent fusion or covalent linkage between an enzyme of interest, such as glucose-6-phosphate dehydrogenase and an analyte.

“Enzyme substrate” means a substrate for an enzyme, e.g., glucose-6-phosphate (G6P) is a substrate for G6PDH.

“Excipient” refers to an inert substance used as a diluent.

“G6PDH” refers to the enzyme glucose-6-phosphate dehydrogenase, which may be obtained either from natural sources, such as from yeast, bacteria, or fungi, in native or mutational form, or prepared by recombinant methods. As used herein, “G6PDH” includes allelic variations normally found in the natural population and changes introduced by recombinant techniques. Included within this definition are proteins and polypeptides that are functionally defined by converting glucose-6-phosphate and NAD (or NADP) to 6-P-glucuronate and NADH (or NADPH). Those of skill in the art recognize that G6PDH proteins or G6PDH polypeptides can be modified in a variety of ways including the addition, deletion and substitution of amino acids.

“Hapten” means a modified drug or analyte with a proper functional group so that it can be covalently linked to desirable proteins to form an immunogen or an enzyme conjugate, etc.

“Illicit drug” refers to a substance, compound or composition for which the production, possession, use, or supply is unlawful.

“Inhibition” refers to the capability of an antibody or receptor to inhibit the activity of an enzyme or an enzyme-analyte conjugate upon binding an epitope present on the analyte.

“Ligand” refers to any organic compound for which a receptor naturally exists or can be prepared.

Synonyms for the phrase “measuring the amount of an analyte” are contemplated within the scope of the present invention and include, but are not limited to, detecting, measuring, or determining an analyte; detecting, measuring, or determining the presence an analyte; detecting, measuring, or determining the amount of an analyte;

“Milli-absorbant unit” or “mA” means one thousandth absorbent unit.

“Licit drug” refers to a substance, compound or composition for which the production, possession, use, or supply is lawful.

“Linking group” refers to a portion of a structure which connects 2 or more substructures. A linking group has at least one uninterrupted chain of atoms extending between the substructures.

“NAD” or “NAD⁺” refers to nicotinamide-adenine dinucleotide, a co-enzyme for G6PDH.

“NADH” refers to reduced nicotinamide adenine dinucleotide. It can be measured by monitoring the absorption in a spectrophotometer at a wavelength of 340 nm, i.e., the characteristic absorption region of NADH.

“NADP” or “NADP⁺” refers to nicotinamide-adenine dinucleotide phosphate, a co-enzyme for G6PDH.

“NADPH” refers to reduced nicotinamide adenine dinucleotide phosphate.

G6PDH from a “natural source” or a “naturally occurring” G6PDH refers to a G6PDH purified from a natural source, including, but not limited to, bacterial, yeast, fungal, vertebrates and mammals.

“Oral fluid” means a biological fluid, such as saliva, obtained from an oral area of an individual.

“Receptor” refers to any compound or composition capable of recognizing a particular spatial and polar organization of a molecule, i.e., an epitope or determinant site on a ligand.

“Receptor reactive to analyte” means that the receptor has an area on its surface or in a cavity which specifically binds to a particular analyte, i.e., it has a binding affinity (usually expressed as Ka) for the analyte.

“Recombinant enzyme” or “recombinant G6PDH” refers to an enzyme (or G6PDH) generated by recombinant DNA technologies, wherein the DNA encoding the enzyme (or G6PDH) is introduced into a host suitable for expression of such DNA and wherein the enzyme (or G6PDH) protein produced by such host is purified.

“Sensitivity” is used in the sense of detection limit, i.e., the smallest amount of an analyte giving a signal that is distinguishable from the signal obtained in the absence of an analyte.

“Signal producing system” refers to a system generating a signal that relates to the presence or amount of an analyte. A signal producing system has at least two components: (1) a catalytic component and (2) a substrate component, which undergoes a reaction catalyzed by the catalytic component and leading directly or indirectly to a product, which generates a detectable signal. The catalytic member may be enzymatic or non-enzymatic, preferably enzymatic, such as G6PDH. The signal generating compound will provide an electromagnetic signal, e.g., a spectrophotometric or visible, electrochemical or electronic detectible signal.

“Starting specific activity” means the enzymatic activity of a natural or recombinant enzyme, such as G6PDH, which is not conjugated or linked to an analyte.

### Incorporation by Reference

All publications and patent applications cited in this specification are herein incorporated by reference as if each individual publication or patent application were specifically and individually indicated to be incorporated by reference.

### DETAILED DESCRIPTION OF THE INVENTION

The present invention provides a signal producing system that generates a signal that relates to the presence or amount of an analyte in a sample suspected of containing an analyte. The present invention shows some similarity to those signal producing systems previously described for testing urine, serum or plasma specimen. However, the signal producing system of this invention differs from those in its increased sensitivity which makes it useful in applications such as identifying analytes in oral fluid where analytes may be present in low concentrations.

For purpose of the present invention, the signal producing system includes at least one enzyme and at least one substrate, and may include two or more enzymes and a plurality of substrates. In particular, the invention provides a homogeneous enzyme immunoassay system for determining the amount of analytes in an oral fluid sample. In general, the immunoassay of the invention works as follows: G6PDH, is provided and its starting specific activity is determined (by measuring the NADH or NADPH produced by G6PDH) or provided by a commercial supplier of G6PDH. G6PDH converts nicotinamide adenine dinucleotide (NAD⁺) or nicotinamide adenine dinucleotide phosphate (NADP⁺) to NADH or NADPH, respectively, resulting in an absorbance change that can be measured spectrophotometrically at 340 nm. Next, the G6PDH is covalently linked to an analyte, resulting in an G6PDH-analyte conjugate. The enzymatic activity of G6PDH of the G6PDH-analyte conjugate is decreased due to the covalent linkage of analyte. This decrease in enzymatic activity is referred to as ‘deactivation.’ Next, an antibody or a receptor reactive to the analyte binds to the analyte of the G6PDH-analyte conjugate. Binding of the antibody or receptor leads to an additional decrease of G6PDH activity. This additional decrease is referred to as ‘inhibition,’ to distinguish it from the deactivation. Upon the addition of a sample containing the same analyte linked to G6PDH, some of the antibodies or receptors bound to the G6PDH-analyte conjugate now bind to the free analyte in the sample and release the G6PDH-analyte conjugate leading to an increase in G6PDH activity. This increase is referred to as ‘reversible inhibition.’ Once calibrated, as described herein, the analyte concentration in the sample is measured in terms of increased G6PDH enzyme activity. Thus, the assay is based on competition between the G6PDH-analyte conjugate and the free analyte in the sample for a fixed amount of specific antibody(ies) or receptor(s). The following will describe the individual components and parameters of the homogenous enzyme immunoassay in detail.

### Glucose-6-Phoshate Dehydrogenase (G6PDH)

The invention provides an homogeneous enzyme assay system comprising an enzyme-analyte conjugate, comprising an enzyme and an analyte. In a preferred embodiment of the invention the enzyme is glucose-6-phosphate dehydrogenase (G6PDH). G6PDH may be capable of using both NADP⁺ and NAD⁺, such as those isolated from Leuconostoc mesenteroides, A. suboxydans, P. aeruginosa, Pseudomonas W6, H. eutrophaH-16, Hydrogenomonas facilis, Arthrobacter 7C, A. beijerickii, T. ferrooxidans, B. licheniformis, P. denitrificans, C. crescentus, L. lactis, and R. spheroides. Alternatively, G6PDH may be capable of using NAD⁺ as a preferred cofactor such as those isolated from P. fluorescens and one of the G6PDHs from P. multivorans, or may be NAD⁺ specific such as one of the G6PDHs from A. xylinum.

For example, Leuconostoc mesenteroides glucose-6-phosphate dehydrogenases are dimeric enzymes that have the ability to catalyze the oxidation of D-glucose-6-phosphate to D-glucono-δ-lactone-6-phosphate by utilizing either NAD⁺ or NADP⁺. This property of using NAD⁺ differentiates these enzymes from human G6PDH, which utilizes only NADP⁺ effectively, and allows L. mesenteroides-specific G6PDH activity to be measured in the presence of human G6PDH, as for example in human samples. Glucose-6-phosphate dehydrogenases from L. mesenteroides are used in current EMIT homogeneous immunoassays (EMIT is a Trademark of Syva Company (now Dade-Behring), Palo Alto, Calif., U.S.A.).

Two preferred genera of bacteria from which to select DNA encoding G6PDH are Leuconostoc and Zymomonas. Within these genera L. mesenteroides, L. citreum, L. lactis, L. dextranicum, and Z. mobilis are preferred, L. mesenteroides, L. citreum, L. lactis being particularly preferred. Because G6PDH from Leuconostoc does not contain cysteine residues, it is preferred for mutation strategies wherein one or more cysteine residues are introduced.

Table 1 of U.S. Pat. No. 6,033,890, the disclosure of which is incorporated in its entirety, describes exemplary strains of various Leuconostoc species. Such strains are purely exemplary and do not limit the selection of G6PDH for use within the context of the present invention to that of any particular genus, species or strain. Among the most preferred strains from which to select G6PDH are Leuconostoc mesenteroides strain ATCC 12291, Leuconostoc citreum strain NCIMB 3351, Leuconostoc lactis strain NCDO 546, and Leuconostoc dextranicum strain ATCC 19255.

Other G6PDH suitable for use in the present invention include, but are not limited to those described in Bacillus megaterium M1286 (Heilman et al., Eur. J. Biochem. (1988) vol. 174, 485-490); Saccharomyces cerevisiae (Jeffrey et al., Biochemistry, (1985) vol. 24, 666-671); Pichia jadinii (Jeffrey et al., Biochem. Biopys. Res. Comm., (1989) vol. 160:3, 1290-1295), E. coli K-12 (Rowley et al., J. Bacteriol., (1991) vol. 173:3, 968-977), and from human (Bhadbade et al., FEBS Lett. (1987) vol. 211, 243-246).

In the homogeneous enzyme immunoassay of this invention suitable to measure low analyte concentration in an oral fluid sample, several factors need to be considered in order to achieve the sensitivity of the assay described herein. These factors include: (1) the starting specific activity of the native G6PDH (i.e., before conjugation to an analyte), (2) the enzymatic activity of the enzyme-analyte conjugate (i.e., % deactivation), (3) the affinity (Ka) of the antibody or receptor to the analyte, (4) the activity of the enzyme-analyte conjugate with bound antibody or receptor reactive to the analyte (i.e., % inhibition), (5) the activity of the enzyme-analyte conjugate with released antibody or receptor (i.e., % reversible inhibition due to competition of antibody or receptor binding to the free analyte in the sample), (6) dilution of oral fluid sample, (7) buffer ingredients, and (8) oral fluid sample volume.

Generally, the higher the starting specific activity of the native G6PDH is, i.e., before conjugation to an analyte, the higher the assay sensitivity will be. It is an objective of this invention to provide G6PDH with a minimum starting specific activity. Thus, in one embodiment of the invention, the starting specific activity of G6PDH is in the range of about 500 units/mg and 2,000 units/mg, preferably in the range of about 600 units/mg and 1,500 units/mg and more preferably in the range of about 700 units/mg and 1,000 units/mg. In one preferred embodiment of the invention, the G6PDH has a starting specific activity of at least about 800 units/mg. In another preferred embodiment, the G6PDH has a starting specific activity of at least about 900 units/mg.

### G6PDH Substrates

The enzymatic activities of G6PDH, the G6PDH-analyte conjugate, the G6PDH-analyte conjugate with bound antibody and the G6PDH-analyte conjugate with bound antibody competing for analyte binding in a test sample are determined. Determination of enzymatic activity is dependent on a substrate and co-enzyme for G6PDH. A suitable substrate for G6PDH is glucose-6-phosphate (G6P). Suitable co-enzymes or cofactors for G6PDH are NAD (NAD⁺) and NADP (NADP⁺). G6PDH converts G6P and co-enzymes into 6-P-glucuronate and NADH and NADPH, respectively. Thus, generally, in order to measure G6PDH activity, G6P and NAD⁺ or NADP⁺ are added to the assay. Cofactor analogs, such as thio-NAD⁺, thio-NADH, thio-NADP⁺, or thio-NADPH may also be used.

Typically, substrate and co-enzyme or co-factors for G6PDH are not labeled and the signal generated by G6PDH, i.e., the amount of NADPH or NADH, is measured in a spectrophotometer as described herein. Alternatively, the substrate and or co-enzymes may be labeled and the signal generated by G6PDH may be detected by other means, depending on the label, such as a fluorometer or scintillation counter, or the like.

### G6PDH from Natural Sources

Different G6PDH enzymes, i.e., G6PDHs from different species, usually display different specific enzymatic activities. It is an objective of this invention to provide G6PDH with a minimum starting specific activity. Thus, in one embodiment of the invention, the starting activity of G6PDH is in the range of about 500 units/mg and 2,000 units/mg, preferably in the range of about 600 units/mg and 1,500 units/mg and more preferable in the range of about 700 units/mg and 1,000 units/mg. In one preferred embodiment of the invention, the G6PDH has a starting specific activity of at least about 800 units/mg. In another preferred embodiment, the G6PDH has a starting specific activity of at least about 900 units/mg.

In order to provide G6PDH enzymes having a desired starting specific activity, the present invention contemplates the use of G6PDH from natural or recombinant sources or site-directed mutants, and any isoform, site-directed mutant or a mixture of isoforms and site-directed mutants may be used.

Several G6PDH enzymes from various species are known as described herein, in U.S. Pat. No. 6,033,890 and by Levy (Adv. Enzym. (1979) vol. 48, 97-192). G6PDH from natural sources may be purified following procedures known to the skilled artisan.

### G6PDH from Recombinant Sources

In one embodiment of the invention, the G6PDH is a recombinant G6PDH. The basic molecular biological techniques employed in generating a recombinant G6PDH, i.e., methods such as DNA and plasmid isolation, restriction enzyme digestion, DNA ligation, purification and characterization of DNAs by polyacrylamide and agarose gel electrophoresis, labeling and hybridization of DNAs, Southern blotting, transformation, maintenance and growth of bacterial strains, protein expression and protein purification, and other general techniques are all well known in the literature. Specifically, the general techniques of molecular biology are described in “Molecular Cloning A Laboratory Manual” by Sambrook, J., Fritsch, E. F., and Maniatis, T. published by Cold Spring Harbor Laboratory Press, 2nd edition, 1989, or “A Practical Guide to Molecular Cloning” by Bernard Perbal published by John Wiley & Sons, New York, 1984.

Generally, the DNA encoding a G6PDH of interest is cloned into an expression vector and transformed into a suitable host cell, which expresses the recombinant G6PDH. The recombinant G6PDH may then be purified using methods known to the skilled artisan. Recombinant G6PDH enzymes have been described and include, but are not limited to, G6PDHs from L. mesenteroides (Adams et al., J. Biol. Chem., (1983) vol. 258:9, 5867-5868; Murphy et al., J. Bacteriol., (1987) vol. 169:1, 334-339; Lee et al., J. Biol. Chem., (1991) vol. 266:20, 13028-13034); Z. mobiles (Barnell et al., J. Bacteriol., (1990) vol. 172:12, 7227-7240); Bacillus megaterium M1286 (Heilmann et al., Eur. J. Biochem., (1988) vol. 174, 485-490); and E. coli K-12 (Rowley et al. J. Bacteriol., (1991) vol. 173:3, 968-977).

### Mutated G6PDH Enzyme

The sensitivity of the present immunoassay with respect to determining the analyte concentration may be modified through the use of different forms of G6PDH. In another embodiment of the invention, the G6PDH is a mutated G6PDH. Thus, G6PDHs differing from any naturally occurring G6PDH may be generated by using molecular DNA cloning technologies as known in the art. G6PDHs with amino acid substitutions, deletions, or insertions, or any combination thereof may be generated (see U.S. Pat. No. 6,033,890) and used in the methods of this invention.

### Commercially Available G6PDH

G6PDH from various sources are also commercially available, e.g., from Sigma, Biochemica, Boehringer Mannheim, USB Biochemical, and OYC International Inc.

### Other Enzymes Suitable for Use in the Present Invention

The invention provides an enzyme-analyte conjugate, comprising an enzyme and an analyte. In a preferred embodiment of the invention, the enzyme is G6PDH. In another embodiment of the invention, the enzyme is an enzyme other G6PDH. Additional enzymes that are useful for the present invention and which use NAD (NAD⁺) as a co-enzyme and generate NADH include alcohol dehydrogenase, glutamic dehydrogenase, malic dehydrogenase, isocitric dehydrogenase, α-glycerol phosphate dehydrogenase, lactic dehydrogenase, and glyceraldehydes-3-phosphate dehydrogenase. Additional enzymes that are useful for the present invention and which use NADP (NADP⁺) as a co-enzyme and generate NADPH include gluthathione reductase, quinine reductase, nitrate reductase, and glutamic dehydrogenase. In addition, a large number of enzymes and co-enzymes useful in the methods of the present invention are disclosed in U.S. Pat. No. 4,275,149 and U.S. Pat. No. 4,318,980, which are incorporated herein by reference. Employing one or more of the above enzymes may further increase the sensitivity of the present immunoassay.

### Analyte

The invention provides an enzyme-analyte conjugate, comprising an enzyme and an analyte. Thus, the analyte can either be linked or conjugated to an enzyme, such as G6PDH, or be free in a sample. An analyte of the invention is any substance, compound or composition whose presence or concentration in a sample or specimen is to be determined.

Analytes can be polyepitopic or monoepitopic. Monoepitopic analytes will generally be from about 100 to 5,000 molecular weight, preferably from 125 to 2,000 molecular weight. Polyepitomic analytes employed in the subject invention will have a molecular weight of at least 5,000 molecular weight, preferably at least about 10,000 molecular weight. Poly amino acid analytes of interest include proteins, polypeptides and peptides and will generally be from about 5,000 to 5,000,000 molecular weight, preferably from about 20,000 to 1,000,000 molecular weight.

The following analytes are contemplated within this invention: licit and illicit drugs, sugars (including, but not limited to, mono-, di-, and poly-carbohydrates), amino acids, peptides, nucleic acids, nucleosides, nucleotides, vitamins, hormones, steroids, antibiotics, bacterial or microbial antigens, toxins, chemical and biological warfare agents, pesticides, herbicides, and industrial chemicals and pollutants. Included in these classes are analogs, derivatives and metabolites of such compounds.

Analyte drugs whose presence or concentration in a sample may be determined using this invention include, but are not limited to, opium, the opioid analgesics, alkaloids, catecholamines, epinephrine, amphetamines, barbiturates, benzodiazepines, cardiac drugs, anti-seizure drugs, immunosuppressants, tetrahydrocannabinol (THC, the active ingredient in marijuana), cocaine, cocaine metabolite (benzoylecgonine), crack, inhalants (e.g., amyl or butyl nitrates), phencyclidine (PCP), 3,4-methylendioxymethamphetamine (MDMA, or ecstasy) and its related compounds such as 3,4-methylendioxyamphetamine (MDA) and 3,4-methylenedioxyethylamphetamine (MDEA), ketamine, lysergic acid diethylamind (LSD), γ-hydroxybutyrate (GHB), methaqualone (also called quinazolinone), tranquilizers, alcohol, etc. Included in these classes are analogs, metabolites, and derivatives of such compounds.

In a preferred embodiment of the invention, the analyte is an opioid analgesic. Opiod analgesics include, but are not limited to, opium, morphine, heroin, codeine, dihydrocodeine (DF-118), hydromorphone, fentanyl, oxycodone, buprenorphine, butorphanol, nalbuphine, methadone, physeptone, pethidine, dioconal, palium, dextromoramide, dipipanone, phenadoxone, propoxyphene (Darvon®), dextroproxyphene, pethidine, methylphenidate (Ritalin), and acetylmethadol. Included in this embodiment are analogs, metabolites, and derivatives of such opioid analgesics.

In another embodiment of the invention, the analyte is an alkaloid. Alkaloids that can be detected using this invention include, but are not limited to, the steroid alkaloids, the iminazolyl alkaloids, the isoquinoline alkaloids, the quinoline alkaloids (including quinine), and the diterpene alkaloids. Included in this embodiment are analogs, metabolites, and derivatives of such alkaloids.

In one embodiment of the invention, the analyte is a catecholamine. Catecholamines include, but are not limited to, cotamine, narceine, noscapine and papaverine epinephrine, L-dopa, and ephedrine. Included in this embodiment are analogs, metabolites, and derivatives of such catecholamines.

In another embodiment of the invention, the analyte is an amphetamine or a related compound. Amphetamines and related compounds include, but are not limited to, amphetamine, methamphetamine, and the like. Included in this embodiment are analogs, metabolites, and derivatives of such amphetamines or related compounds.

In one embodiment of the invention, the analyte is a barbiturate. Barbiturates include, but are not limited to, veronal, pentobarbital (Nembutal), amobarbital, secobarbital (Seconal), phenobarbital, and thiopental, etc. Included in this embodiment are analogs, metabolites, and derivatives of such barbiturates.

In another embodiment of the invention, the analyte is a benzodiazepine. Benzodiazepines include, but are not limited to, Diazepam (Valium), chlordiazepoxide (Librium), Nitrazepam (Mogodon), and Temazepam. Included in this embodiment are analogs, metabolites, and derivatives of such benzodiazepines.

In a preferred embodiment of the invention, the analyte is a hallucinogen. Hallucinogens include, but are not limited to, mescaline, psilocybin, psilocin, dextromoramide (Palfium), LSD, MDA (3,4-methylenedioxyamphetamine), Ecstacy (MDMA, 3,4-methylenedioxymethamptamine), MDEA (3,4-methylenedioxyethylamphetamine), PMA (para-methoxyamphetamine), PMMA (para-methoxymethylamphetamine), PCP (phencyclidine). Included in this embodiment are analogs, metabolites, and derivatives of such hallucinogens.

In another embodiment of the invention, the analyte is a cardiac drug. Cardiac drugs include, but are not limited to, digoxin, digitoxin, N-acetyl procainamide, procainamide, quinidine, and lidocaine. Included in this embodiment are analogs, metabolites, and derivatives of such cardiac drugs.

In one embodiment of the invention, the analyte is an anti-seizure drug. Anti-seizure drugs include, but are not limited to, phenytoin, Phenobarbital, primidone, valproic acid, ethosuximide, and carbamazepine. Included in this embodiment are analogs, metabolites, and derivatives of such anti-seizure drugs.

In another embodiment of the invention, the analyte is an immunosuppressant. Immunosuppressant include, but are not limited to, MPA (mycophenolic acid), cyclosporine, rapamycin (sirolimus), and FK506 (tacrolimus). Included in this embodiment are analogs, metabolites, and derivatives of such immunosuppresants.

Additional analytes contemplated by this invention are vitamins and diet supplements such as folic acid, thiamine, Vitamin B₁₂, biotin, Vitamin A, Vitamin B, Vitamin C, Vitamin D, Vitamin E, Vitamin K, tranquilizers such as meprobamate, and tricyclic anti-depressants, food supplements and other performance-enhancing agents. Included in this embodiment are analogs, metabolites, and derivatives of such compounds.

In another embodiment of the invention, the analyte is an amino acid. Amino acids whose presence may be detected include, but are not limited to, glycine, alanine, serine, histidine, and methionine. Included in this embodiment are analogs, metabolites, and derivatives of such amino acids.

In one embodiment, the analyte is an antibiotic. Antibiotics include, but are not limited to, penicillin, chloromycetin, actinomycin, tetracycline, terramycin, gentamycin, kanamycin, tobromycin, tobramycin, netilmicin, amikacin, and vancomycin. Included in this embodiment are analogs, metabolites, and derivatives of such antibiotics.

In another embodiment of the invention, the analyte is a microbial antigen. Microbial antigens include, but are not limited to, Clostridium difficile antigen, Toxin A, and aflatoxin B₁. Included in this embodiment are analogs, metabolites, and derivatives of such microbial antigens.

In another embodiment of the invention, the analyte is a hormone. Hormones, include, but are not limited to, thyroid hormones (T₃ and T₄), thyroxine, thyroid stimulating hormone, estrogen, progesterone, testosterone, prolactin, follicle stimulating hormone, chorionic gonadotropin, and luteinizing hormone. Included in this embodiment are analogs, metabolites, and derivatives of such hormones.

In one embodiment of the invention, the analyte is a steroid. Steroids include, but are not limited to, various estrogens and androgens such as ethynylestradiol, testosterone and androsterone. Included in this embodiment are analogs, metabolites, and derivatives of such steroids.

In another embodiment of the invention, the analyte is a chemical or biological warfare agent. Chemical or biological warfare agents include, but are not limited to, mustard gas, Sarin, Tabun, Bacillus anthracis (Anthrax) antigens, and Smallpox viral antigens. Included in this embodiment are analogs, metabolites, and derivatives of such chemical or biological warfare agents.

In one embodiment of the invention, the analyte is an industrial chemical. Industrial chemicals include, but are not limited to, flavoring agents, food additives, preservatives, food contaminants, air and chemical pollutants, pesticides, and herbicides. Included in this embodiment are analogs, metabolites, and derivatives of such industrial chemicals.

### Conjugation

The invention provides an enzyme-analyte conjugate comprising an enzyme covalently linked or conjugated to an analyte. In a preferred embodiment, G6PDH is conjugated to the analyte resulting in a G6PDH-analyte conjugate. Conjugation reactions with enzymes, such as G6PDH, can be affected by a number of factors. These include, but are not confined to, pH, temperature, buffer, ionic strength, substances which may protect the enzyme active site, amount and type of cosolvent, reaction time, and activation chemistry. Appropriate manipulation of these variables can lead to G6PDH-analyte conjugates which are improved in one or more of the following properties: 1) reduced deactivation; 2) larger standard curve; 3) improved assay precision; or 4) enhanced thermal stability.

Conjugation can be achieved via conventional chemical reactions as known in the art. Among them, the simplest reaction to coupling an analyte (or a hapten) to G6PDH is through the formation of a peptide bond (—CONH₂). For example, using a carboxyl (—COOH) group on an analyte (or a hapten) to react with an amino group (—NH₂) on the G6PDH enzyme (Biochem. and Biophys. Res. Comm., (1989) vol. 160:3, 1290-1295). Glucose-6-phosphate dehydrogenase from Leuconostoc mesenteroides is reported to contain a total of 38 lysine residues (Levy, Adv. Enzym, (1979) vol. 48, 97-192; FEBS Lett. 211:2, 243-246, 1987). Under appropriate coupling conditions, the ε-amino groups from these lysine moieties can be modified readily. Therefore multiple molecules of an analyte (or hapten) and/or a plurality of analytes (or haptens) can be conjugated to each molecule of G6PDH.

Some analytes are capable of binding directly to G6PDH. Others are not capable of covalent binding directly. Such analytes are rendered capable of covalently binding to G6PDH by the addition of a linking group (i.e, definition of haptens) that can covalently bind to a group on G6PDH (for instance, to an amino, hydroxyl, carboxyl or mercapto group). Such linking groups may comprise, for instance, amino acids having one or more free amino or free hydroxyl groups, or may comprise carbonyl, thiocarbonyl, or carboxyl groups, or compounds containing such groups. Linking groups commonly used for this purpose include N-hydroxysuccinimide and other succinimide or maleimide-containing moieties, and 1-(3-dimethylpropyl)-3-ethylcarbodiimide. A detailed discussion of such linking groups is found in U.S. Pat. No. 3,817,837, which is incorporated by reference in its entirety.

Linking groups suitable for use in this invention include, but are not limited to compounds of less than 50 atoms other than hydrogens, preferably less than 20 atoms other than hydrogens, more preferably less than 6 atoms other than hydrogens and having a chain (i.e., a spacer) of usually not more than 35, preferably less than 15, more preferably less than 10, and most preferably less than 5 atoms in length.

For example, linking groups usable in preparing conjugates for this invention include bifunctional crosslinking or coupling agents, i.e., molecules containing two reactive groups or “ends”, which may be tethered by a spacer of variable length. The reactive ends can be any of a variety of functionalities including, but not limited to, amino reacting ends such as N-hydroxysuccinimide (NHS) active esters, imidoesters, aldehydes, epoxides, sulfonyl halides, isocyanate, isothiocyanate, and nitroaryl halides; and thiol reacting ends such as pyridyl disulfides, maleimides, and thiophthalimides. The heterobifunctional crosslinking reagents have two different reactive ends, e.g., an amino-reactive end and a thiol-reactive end, while homobifunctional reagents that are usable in preparing the conjugates of this invention have two similar reactive ends. Examples of such include bismaleimidohexane (BMH), which permits the cross-linking of sulfhydryl-containing compounds, and NHS homobifunctional crosslinkers such as disuccinimidyl suberate (DSS) as well as the water soluble analogs, sulfo-NHS esters.

Other suitable linking groups for use in the present invention include, but are not limited to, maleimido-NHS active esters coupling agents such as m-maleimidobenzoyl-N-hydroxy-succinimide ester (MBS); succinimidyl 4-(N-maleimidomethyl)cyclohexane-1-carboxylate (SMCC); succinimidyl 4-(p-maleimidophenyl)butyrate (SMPB) and derivatives thereof, including sulfosuccinimidyl derivatives such as sulfosuccinimidyl 4-(N-maleimido-methyl) cyclohexane-1-carboxylate (sulfo-SMCC); m-maleimidobenzoyl-sulfosuccinimide ester (sulfo-MBS) and sulfosuccinimidyl 4-(p-maleimidophenyl)butyrate (sulfo-SMPB) (Pierce). Other suitable heterobifunctional reagents include commercially available active halogen-NHS active esters coupling agents such as N-succinimidyl bromoacetate and N-succinimidyl (4-iodoacetyl)aminobenzoate (SIAB) and the sulfosuccinimidyl derivatives such as sulfosuccinimidyl(4-iodoacetyl)aminobenzoate (sulfo-SIAB) (Pierce). Another group of coupling agents is the heterobifunctional and thiol cleavable agents such as N-succinimidyl 3-(2-pyridyidithio)propionate (SPDP) (Pierce).

Other commercially available homobifunctional cross-linking reagents include, but are not limited to, the imidoesters such as dimethyl adipimidate dihydrochloride (DMA); dimethyl pimelimidate dihydrochloride (DMP); and dimethyl suberimidate dihydrochloride (DMS).

The choice of the amine-reactive modification reagent, thiol introducing agent or other activating agent is not critical, but one skilled in the art will know of suitable or preferred agents for use with the particular analyte whose presence in the sample is to be determined. Therefore, the linking group to be used will generally be determined empirically.

The conjugates are prepared by contacting the activated analyte or hapten with a buffered solution of G6PDH under typical conditions for formation of such conjugates. Typical conditions for forming such conjugates include a temperature of from about 2° C. to about 25° C., a pH of from about 5 to about 10, and a contact time of from less than an hour to several days.

After the conjugation, the enzyme-analyte conjugate may be purified. Suitable purification procedures are known in the art and include dialysis against aqueous/organic and aqueous solutions such as water/DMF or water, or by gel filtration chromatography on supports such as Sephadex, and the like.

### Deactivation of G6PDH

Usually, covalently linking an analyte to G6PDH leads to a change of G6PDH enzymatic activity, which can be measured using the methods of this invention. Generally, this change of enzymatic activity is a decrease of enzymatic activity by the G6PDH-analyte conjugate when compared to the activity of the native G6PDH, i.e., the G6PDH, which is not conjugated to an analyte. The decrease of enzymatic activity due to the covalent linking of an analyte to the G6PDH is referred to as deactivation.

The ratio of analyte conjugated to G6PDH is generally dependent on the desirable % of deactivation of the G6PDH and the desirable % inhibition of the resulting G6PDH-analyte conjugate exhibited upon binding to specific antibody or receptor (as is described herein). Typically, the extent of deactivation will be proportional to the extent of conjugation. The extent of deactivation may be controlled, for example, by measuring enzymatic activity on samples taken at various times of conjugation.

In one embodiments of the invention, the G6PDH is deactivated by from about 20% to about 60% and the enzyme activity of the deactivated G6PDH-analyte conjugate is further inhibited by from about 40% to about 80%.

In another embodiment of the invention, the G6PDH is deactivated by from about 30% to about 65% and the enzyme activity of the deactivated G6PDH-analyte conjugate is further inhibited by from about 40% to about 85%.

Generally, the higher the specific activity of the G6PDH-analyte conjugate, the higher the assay sensitivity will be. It is an objective of this invention, to provide a G6PDH-analyte conjugate with a minimum specific activity.

Thus, with about 10% deactivation, in one embodiment of the invention, the specific activity of the G6PDH-analyte conjugate is in the range of about 450 units/mg and 1,800 units/mg, preferably in the range of about 540 units/mg and 1,350 units/mg and more preferably in the range of about 630 units/mg and 900 units/mg. In one preferred embodiment of the invention, the G6PDH-analyte conjugate has a specific activity of at least about 720 units/mg. In another preferred embodiment, the G6PDH-analyte conjugate has a specific activity of at least about 810 units/mg.

Thus, with about 20% deactivation, in one embodiment of the invention, the specific activity of the G6PDH-analyte conjugate is in the range of about 400 units/mg and 1,600 units/mg, preferably in the range of about 480 units/mg and 1,200 units/mg and more preferably in the range of about 560 units/mg and 800 units/mg. In one preferred embodiment of the invention, the G6PDH-analyte conjugate has a specific activity of at least about 640 units/mg. In another preferred embodiment, the G6PDH-analyte conjugate has a specific activity of at least about 720 units/mg.

Thus, with about 30% deactivation, in one embodiment of the invention, the specific activity of the G6PDH-analyte conjugate is in the range of about 350 units/mg and 1,400 units/mg, preferably in the range of about 420 units/mg and 1,050 units/mg and more preferably in the range of about 490 units/mg and 700 units/mg. In one preferred embodiment of the invention, the G6PDH-analyte conjugate has a specific activity of at least about 560 units/mg. In another preferred embodiment, the G6PDH-analyte conjugate has a specific activity of at least about 630 units/mg.

Thus, with about 40% deactivation, in one embodiment of the invention, the specific activity of the G6PDH-analyte conjugate is in the range of about 30 units/mg and 1,200 units/mg, preferably in the range of about 360 units/mg and 900 units/mg and more preferably in the range of about 420 units/mg and 600 units/mg. In one preferred embodiment of the invention, the G6PDH-analyte conjugate has a specific activity of at least about 480 units/mg. In another preferred embodiment, the G6PDH-analyte conjugate has a specific activity of at least about 540 units/mg.

Thus, with about 50% deactivation, in one embodiment of the invention, the specific activity of the G6PDH-analyte conjugate is in the range of about 250 units/mg and 1,000 units/mg, preferably in the range of about 300 units/mg and 750 units/mg and more preferably in the range of about 350 units/mg and 500 units/mg. In one preferred embodiment of the invention, the G6PDH-analyte conjugate has a specific activity of at least about 400 units/mg. In another preferred embodiment, the G6PDH-analyte conjugate has a specific activity of at least about 450 units/mg.

Thus, with about 60% deactivation, in one embodiment of the invention, the specific activity of the G6PDH-analyte conjugate is in the range of about 200 units/mg and 800 units/mg, preferably in the range of about 240 units/mg and 600 units/mg and more preferably in the range of about 280 units/mg and 400 units/mg. In one preferred embodiment of the invention, the G6PDH-analyte conjugate has a specific activity of at least about 320 units/mg. In another preferred embodiment, the G6PDH-analyte conjugate has a specific activity of at least about 360 units/mg.

### Antibodies

In one embodiment of the invention, an antibody binds to the analyte of the enzyme-analyte conjugate. Antibodies contemplated by this invention include one or more polypeptides substantially encoded by immunoglobulin genes or fragments of immunoglobulin genes. Immunoglobulin genes include the kappa, lambda, alpha, gamma, delta, epsilon and mu constant region genes, as well as myriad immunoglobulin variable region genes. Immunoglobulin light chains are classified as either kappa or lambda. Immunoglobulin heavy chains are classified as gamma, mu, alpha, delta, or epsilon, which define immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well-characterized fragments produced by digestion with various peptidases. Thus, for example, pepsin digests an antibody below the disulfide linkages in the hinge region to produce F(ab)₂, a dimer of Fab, which itself is a light chain joined to V_{H}-C_{H1} by a disulfide bond. The F(ab)₂ may be reduced under mild conditions to break the disulfide linkage in the hinge region thereby converting the (Fab)₂ dimer into a Fab monomer. The Fab monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab fragments may be synthesized de novo either chemically or by utilizing recombinant DNA methodology.

Thus, the term antibody, as used herein also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked V_{H}-V_{L} heterodimer, which may be expressed from a nucleic acid including V_{H}- and V_{L}-encoding sequences either joined directly or joined by a peptide-encoding linker (Huston, et al. (1988) Proc. Natl. Acad. Sci. USA, 85: 5879-5883). While the V_{H} and V_{L} are connected to each as a single polypeptide chain, the V_{H} and V_{L} domains associate non-covalently. The first functional antibody molecules to be expressed on the surface of filamentous phage were single-chain Fv's (scFv), however, alternative expression strategies have also been successful. For example, Fab molecules can be displayed on phage if one of the chains (heavy or light) is fused to g3 capsid protein and the complementary chain exported to the periplasm as a soluble molecule. The two chains can be encoded on the same or on different replicons; the important point is that the two antibody chains in each Fab molecule assemble post-translationally and the dimer is incorporated into the phage particle via linkage of one of the chains to g3p (see, e.g., U.S. Pat. No. 5,733,743). The scFv antibodies and a number of other structures converting the naturally aggregated, but chemically separated light and heavy polypeptide chains from an antibody V region into a molecule that folds into a three dimensional structure substantially similar to the structure of an antigen-binding site are known to those of skill in the art (see e.g., U.S. Pat. Nos. 5,091,513, 5,132,405, and 4,956,778).

Particularly preferred antibodies include all those that have been displayed on phage (e.g., scFv, Fv, Fab and disulfide linked Fv (Reiter et al. Protein Eng., (1995) vol. 8, 1323-1331). Antibodies can also include diantibodies, miniantibodies, humanized antibodies, or chimeric antibodies.

The binding constant of an antibody to a non-complementary antigen, for example, the binding constant for amphetamine binding to the antibody for methamphetamine or the binding constant for methamphetamine binding to an antibody for amphetamine, is less than 10% of the binding constant of the antibody for its complementary antigen.

Generally, the binding of analyte (antigen) to antibody in the homogeneous enzyme immunoassay is reversible. The reversible binding reaction between an antigen with a single antigenic determinant (denote Ag) and a single antigen-binding site (denoted Ab) can be expressed as

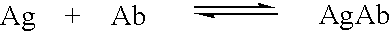

The strength of the binding interaction is generally expressed as the affinity constant (Ka), where Ka=[AgAb]/[Ag][Ab].

For homogeneous enzyme immunoassays, in order to obtain a modulable signal from the enzyme-analyte conjugate (i.e., an enzymatic activity that can be further inhibited), typically, the concentration of each component is at equilibrium, i.e., half the binding sites are filled, such that [AgAb]=[Ab], and half of the antibody (unbound) can bind to the antigen-enzyme conjugate. Thus at the equilibrium point, Ka=1/[Ag]. The reciprocal of the antigen concentration that produces half-maximal binding is equal to the affinity constant of the antibody for the antigen. Common values of affinity constant range from 5×10⁴ to as high as 10¹¹ liter/mole (M⁻¹).

The desirable antibody affinity, calculated based on half of the SAMHSA desirable detection rage (i.e., after a 1 to 2 dilution of the oral fluid sample) of the analyte in the oral fluid is as follows:

| Analyte | Cutoff value | Ka of Ab | |
|---|---|---|---|
| Amphetamine | 25 ng/ml | ≧5 × 10⁸ | M⁻¹ |
| Cocaine Metabolite | 10 ng/ml | ≧2 × 10⁹ | M⁻¹ |
| Methamphetamine | 25 ng/ml | ≧5 × 10⁸ | M⁻¹ |
| Methadone | 20 ng/ml | ≧10⁹ | M⁻¹ |
| Opiate | 20 ng/ml | ≧10⁹ | M⁻¹ |
| Phencyclidine | 5 ng/ml | ≧10⁹ | M⁻¹ |
| THC | 1 ng/ml | ˜4 × 10¹⁰ | M⁻¹ |

Antibodies according to the above specification can be identified by screening cell lines as known in the art and then selecting the proper antibodies for the oral fluid homogeneous enzyme immunoassay.

Usually, the antibody reactive to the analyte has an affinity for the analyte (Ka) in the range of at least 1×10⁸M⁻¹ to at least 5×10⁸ M⁻¹, more preferably in the range of at least 5×10⁸ M⁻¹ to at least 2×10⁹ M⁻¹, most preferably in the range of at least 2×10⁹ M⁻¹ to at least 5×10⁹ M⁻¹. In a preferred embodiment of this invention, the Ka is ≧x10¹⁰M⁻¹. In another preferred embodiment of this invention, the Ka is ≧1×10¹¹M⁻¹.

Antibodies can be prepared by techniques that are well known in the art. Polyclonal antibodies can be prepared by injecting an antigen, such as an analyte, into a wide variety of vertebrates in accordance with conventional methods for the production of antibodies. Likewise, monoclonal antibodies useful in this invention may be produced according to hybrid cell line technologies. Antibodies specific to analytes can be obtained from commercial sources, such as Cortex Biochem, Inc., Biodesign International, and Fitzgerald Industry, Inc.

### Receptors

In one embodiment of the invention, a receptor is used to bind to the analyte. The enzyme-analyte conjugate, more specifically, the G6PDH-analyte conjugate, is mixed with a receptor that is specifically reactive to both the analytes within the G6PDH-analyte conjugate and the free analyte. The receptor can be any composition that can bind effectively and specifically to an analyte and when bound to an enzyme-analyte conjugate cause an inhibition of the activity of the enzyme, such as G6PDH. Suitable receptors would include, but are not limited to, soluble forms of natural receptors to ligand/analytes such as lectins (e.g., for carbohydrates), opioid receptors (e.g., for morphine and opioid peptides), hormone binding receptors (e.g., for hormones), steroid receptors (e.g., for steroids), receptors for enzymes (e.g., for substrates, inhibitors, or cofactors), receptors for intrinsic factors (e.g., Vitamin B₁₂ and other vitamins), etc.

### Inhibition of G6PDH-Analyte Conjugate by Antibody/Receptor

It is an objective of this invention to further inhibit the enzymatic activity of the deactivated enzyme-analyte conjugate, more specifically, the G6PDH-analyte conjugate, by binding of an antibody or receptor reactive to the analyte in the enzyme-analyte conjugate, more specifically, the G6PDH-analyte conjugate.

The extent of analyte conjugation to the G6PDH is proportional to the % inhibition by the antibody or receptor reactive to the analyte in the G6PDH-analyte conjugate. Generally, the more analyte is conjugated to the G6PDH, the more antibody or receptor can bind to the analyte and the higher the % inhibition will be.

Preferably, the deactivated G6PDH-analyte conjugate is further inhibited from about 40% to about 85%. In other words, the antibody or receptor bound G6PDH-analyte conjugate has about 15% to about 60% of specific activity of the G6PDH-analyte conjugate.

In another embodiment of the invention, the deactivated G6PDH-analyte conjugate is further inhibited from about 30% to about 65%. In other words, the antibody or receptor bound G6PDH-analyte conjugate has about 35% to about 70% of the specific activity of the G6PDH-analyte conjugate.

In another embodiment of the invention, the deactivated G6PDH-analyte conjugate is further inhibited from about 40% to about 75%. In other words, the antibody or receptor bound G6PDH-analyte conjugate has about 25% to about 60% of the specific activity of the G6PDH-analyte conjugate.

Preferably, the deactivated enzyme-analyte conjugate is further inhibited from about 60% to about 80%. In other words, the antibody or receptor bound enzyme-analyte conjugate has about 20% to about 40% of the specific activity of the G6PDH-analyte conjugate.

It is preferable that % inhibition/% deactivation is >1, preferably >2, more preferably >3, more preferably >4, and most preferably >5.

### Oral Fluid Sample Preparation

Any sample that is reasonably suspected of containing an analyte of interest can be analyzed using the method of the present invention. In a preferred embodiment of this invention, the sample is a bodily fluid. In a more preferred embodiment, the sample is an oral fluid sample, such as saliva. The oral fluid sample may be collected from an individual and analyzed shortly thereafter using the methods of the present invention. Alternatively, an oral fluid sample may be an oral fluid sample with added preservatives as known in the art.

Sometimes, due to its viscosity and the presence of some insoluble substances, the saliva, as collected from an individual, may not be suitable for direct use as a specimen for analysis. Thus, in one embodiment of the invention, the oral fluid sample is pretreated to ensure accurate and reliable determination of analytes in the sample to be tested. Pretreatment makes oral fluid suitable to be analyzed in most commonly used analyzer. Pretreatment may include dilution of the oral fluid with a buffer followed by filtration and/or centrifugation. Typically, pretreatment provides a clear specimen. With respect to pretreatment of oral fluid, four important aspects are considered and be defined for immunoassay performance and accuracy. These are (1) dilution factor, (2) dilution buffer, (3) buffer compositions, and (4) sample volume.

### Dilution of Oral Fluid Sample

The invention provides for the adjustment of homogeneous enzyme immunoassay sensitivity. In a preferred embodiment of this invention, the sensitivity is adjusted by diluting the sample suspected of containing an analyte, in particular an oral fluid sample suspected of containing an analyte. The dilution factor and the dilution buffer components need to be evaluated to ensure accurate and reliable performance of the homogeneous enzyme immunoassays using oral fluid samples.

As described herein, SAMHSA's guidelines for oral fluid cutoff concentration for certain analytes, such as abused drugs, are much lower than the guidelines for the same analytes in urine specimens. After dilution of the oral fluid sample, the cutoff concentration for those analytes will be even lower. Most currently available oral fluid collectors use a 1 to 4 dilution (e.g., 1 ml of oral fluid+3 ml of dilution buffer), which often causes the analyte concentration in the resulting samples to fall below the detecting limit of currently used assays. It is an object of the invention to provide a method for pretreatment of an oral fluid sample suspected of containing an analyte. In one embodiment of the invention, the oral fluid sample is diluted 1 to 1 (e.g., 1 ml of oral fluid+1 ml of dilution buffer). In another embodiment of the invention, the oral fluid sample is diluted 1 to 0.5 (e.g., 1 ml of oral fluid+0.5 ml of dilution buffer). Using these pretreatment methods, usually, the specimen is within the sensitivity of the homogeneous enzyme immunoassay.

### Buffering the Oral Fluid Sample

The invention provides for the adjustment of homogeneous enzyme immunoassay sensitivity. In a preferred embodiment of this invention, the sensitivity is adjusted by adding a buffer to the sample suspected of containing an analyte, in particular to an oral fluid sample suspected of containing an analyte.

The pH and components of the dilution buffer need to be evaluated to ensure accurate and reliable performance of the homogeneous enzyme immunoassays using oral fluid samples. Saliva has a pH range from between 6.2 and 7.4. Currently, available oral fluid collectors provide a dilution buffer with a pH lower than that of normal saliva. As a result of this procedure, most samples will not be suitable for accurate and reliable analysis using the homogeneous enzyme immunoassay. Thus, it is an object of this invention to provide an oral fluid dilution buffer, which when used to dilute an oral fluid sample, results in an oral fluid sample suitable for accurate and reliable analysis using a homogeneous enzyme immunoassay. In a preferred embodiment of the invention, the oral fluid buffer has a buffer capacity of between 80 mM and 100 mM and a pH in the range from between 7.0 and 9.0. In another embodiment of this invention, the pH of the oral fluid buffer is in the range from between 7.5 and 8.5. In one embodiment of the invention, the pH of the oral fluid buffer is in the range from between 7.5 and 8.2.

Using the oral fluid buffer of this invention, oral fluid samples can be adjusted to a final pH range from between 7.2 and 7.8, which is suitable for homogeneous enzyme immunoassays. Usually the pH of the oral fluid sample will be buffered to a pH range from 4.0 to 11.0, more usually from between 5.0 and 10.0, preferably from between 6.0 and 9.0, more preferably from between 7.0 and 8.5, most preferably from between 7.2 and 8.3. In one embodiment, the oral fluid sample will be buffered to a pH range from between 7.2 and 7.8. A skilled artisan understands that the pH of the enzymatic reaction may be adjusted depending on the particular G6PDH enzyme used in the homogeneous enzyme immunoassay and, accordingly, will adjust the pH to a range in which the G6PDH enzyme has its greatest enzymatic activity.

Various buffers may be used to achieve the desired pH and maintain the desired pH during most homogeneous enzyme immunoassays. Illustrative buffers include borate, phosphate, carbonate, tris, barbital and the like. However, not all buffers are suitable analyzing the low analyte concentration in oral fluid samples. Particularly, some of these buffer ingredients are not desirable for homogenous enzyme immunoassays employing G6PDH. Thus, it is an objective of this invention to provide components of an oral fluid buffer suitable for the analysis of an oral sample suspected of containing an analyte. In a preferred embodiment of this invention, the oral fluid buffer contains tris (Tris-(hydroxymethyl)-aminomethane. Usually, the final concentration of tris in the homogeneous enzyme immunoassays of this invention is in the range of 50-200 mM, preferably in the range of 75-150 mM, more preferably in the range of 80-100 mM. Using the oral fluid buffer of this invention, oral fluid samples can be adjusted to a final pH range from between 7.2 to 8.3, which is suitable for homogeneous enzyme immunoassays using G6PDH.

### Oral Fluid Sample Size

The invention provides for the adjustment of homogeneous enzyme immunoassay sensitivity. In a preferred embodiment of this invention, the sensitivity is adjusted by adjusting the volume of the sample suspected of containing an analyte, in particular an oral fluid sample suspected of containing an analyte. The volume of the oral fluid sample used in the methods of the invention need to be evaluated to ensure accurate and reliable performance of the homogeneous enzyme immunoassays using oral fluid samples. As described herein, the signal generated by the G6PDH-analyte conjugate is proportional to the modulation of the enzyme activity, i.e., reversible inhibition of the antibody. The antibody binding reversibility is proportional to the amount of analyte present in the sample. Thus, typically, increasing the sample size will enhance the % of reversible inhibition. Based on the considerations of buffer dilution, antibody affinity, deactivation and inhibition of enzyme-analyte conjugates and confirmed by experimental results, it was discovered that the sample volume is an important factor for accurate and reliable assay performance. The importance of the sample volume is clearly illustrated by the calculations herein and Examples 4, 8, 9, 10, and 11.

Thus, it is an objective of this invention to provide a sample size of an oral fluid sample to be analyzed for the presence or amount of an analyte. Using currently available commercial analyzers, which use a total assay volume of about 250 μl or more, usually, the sample size of the oral fluid sample is from about 10 μl to about 90 μl, more usually from about 20 μl to about 70 μl, preferably from about 30 μl to about 60 μl, most preferably from about 40 μl to 50 μl. In a preferred embodiment of this invention, the sample size for oral fluid is between about 20 μl and about 50 μl.

It is an objective of this invention to provide homogeneous enzyme immunoassays of oral fluid samples suitable for automatic or semi-automatic analysis in commercially available analyzers. Should commercially available analyzers be used which require a total assay volume of less than 250 μl, then it is an objective of this invention, to adjust the above listed ranges for the oral fluid sample volume accordingly. Thus, by way of example, if an analyzer requires an assay volume of 50 μl, then in one embodiment, the sample size of the oral fluid sample is between about 4 μl and about 10 μl. Typically, the volume of the oral fluid sample will be adjusted for optimal assay performance.

### Filtering the Oral Fluid Sample

The invention provides for the adjustment of homogeneous enzyme immunoassay sensitivity. In a preferred embodiment of this invention, the sensitivity is adjusted by filtering the sample suspected of containing an analyte, in particular an oral fluid sample suspected of containing an analyte.

### Centrifuging the Oral Fluid Sample

The invention provides for the adjustment of homogeneous enzyme immunoassay sensitivity. In a preferred embodiment of this invention, the sensitivity is adjusted by centrifuging the sample suspected of containing an analyte, in particular an oral fluid sample suspected of containing an analyte.

### Stabilizing the Oral Fluid Sample

The invention provides for the adjustment of homogeneous enzyme immunoassay sensitivity. In a preferred embodiment of this invention, the sensitivity is adjusted by adding a stabilizer to the assay medium or the assay components. Such stabilizers may include, but are not limited to, proteins, such as albumin, and surfactants, such as non-ionic surfactants, binding enhancers, e.g., polyalkylene glycols, or the like.

In one embodiment of the invention, a non-ionic detergent as known in the art, is added to the oral fluid sample. Various polyoxyalkylene compounds of from about 200 to 20,000 daltons may be used in the methods of this invention. These compounds may be added to prevent the loss of hydrophobic analytes binding to a sample container.

### Calibration of Homogeneous Enzyme Immunoassay

Due to the nature of reagents, treatments of oral fluid samples, and requirement of the oral fluid volume for the homogeneous enzyme immunoassay, as described herein, a special formulation of calibrators and controls will be needed to validate immunoassay performance and to determine the amount of analyte in an oral fluid sample suspected of containing an analyte.

By comparing the observed G6PDH enzymatic activity obtained by analyzing an oral fluid sample with the G6PDH enzymatic activity obtained from an immunoassay having a known amount of analyte, one can qualitatively and quantitatively determine the analyte of interest in the oral fluid sample analyzed.

Thus, in one embodiment of the invention, calibration components (or calibrators) are provided. The calibration component contains a known amount of the analyte to be determined. For example, the calibration component may comprise analyte samples containing 0, 5, 10, 20, 50, 100, 250, 500, or 1,000 ng/ml of an analyte.

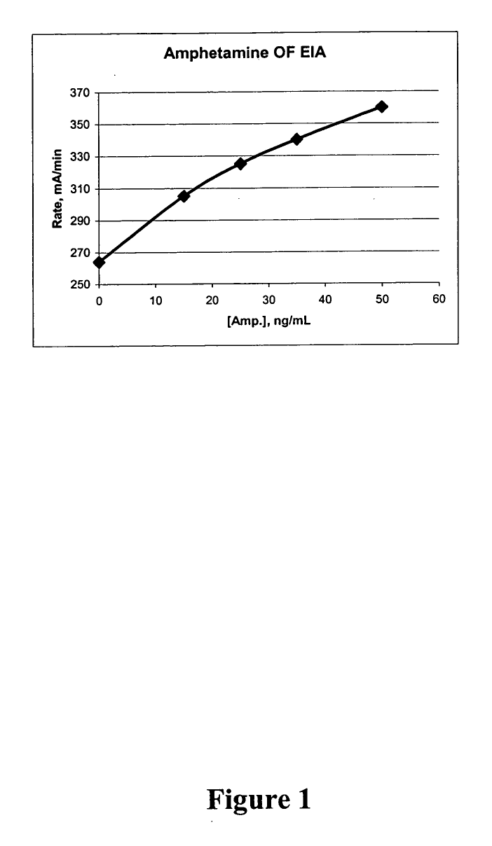

A sample suspected of containing an analyte of interest and the calibration component (or calibrator) containing a known amount of the same analyte are assayed under similar conditions (i.e., similar buffer and sample volumes, etc.) Analyzing the known analyte samples results in a standard calibration curve (see Examples 7, 8, 10, 12, 13, and 14 and FIGS. 1 to 6). The analyte concentration in the sample suspected of containing an analyte of interest is then calculated by comparing the results obtained for the unknown specimen with the results obtained for the standard.

Thus, it is an objective of this invention to provide a formulation buffer for the calibration compound. The buffer may comprise tris buffer, protein, sodium chloride, non-ionic detergent, or sodium azide. Usually, the buffer capacity of the formulation buffer for the calibration compound is in the range of 50-200 mM, preferably in the range of 75-150 mM, more preferably in the range of 80-100 mM.

Likewise, known amounts of analyte may also be added to an oral fluid sample that is clearly negative for the analyte to be measured.

### Homogeneous Enzyme Immunoassay

Any sample which is reasonably suspected of containing an analyte can be analyzed by the methods of the present invention. Although the homogeneous enzyme immunoassays of this invention are useful to identify analytes in any bodily fluid, this invention is particularly useful to identify and determine the amount of an analyte in an oral fluid sample. Thus, in a preferred embodiment, an oral fluid sample suspected of containing an analyte is contacted with an enzyme-analyte conjugate, preferably a G6PDH-analyte conjugate, an, antibody or receptor reactive to the analyte, a substrate for the enzyme (e.g., glucose-6-phosphate and NAD⁺ or NADP⁺ for G6PDH) and a homogeneous competitive enzyme immunoassay is carried out as described herein.

In general, this assay works as follows: G6PDH, is provided and its starting specific activity is determined (by measuring the NADH or NADPH produced by G6PDH) or provided by a commercial supplier of G6PDH. G6PDH converts nicotinamide adenine dinucleotide (NAD⁺) or nicotinamide adenine dinucleotide phosphate (NADP⁺) to NADH or NADPH, respectively, resulting in an absorbance change that can be measured spectrophotometrically at 340 nm. Next, the G6PDH is covalently linked to an analyte, resulting in an G6PDH-analyte conjugate. The enzymatic activity of G6PDH of the G6PDH-analyte conjugate is decreased due to covalent linkage of analyte. This decrease in enzymatic activity is referred to as ‘deactivation.’ Next, an antibody or a receptor reactive to the analyte binds to the analyte of the G6PDH-analyte conjugate. Binding of the antibody or receptor leads to an additional decrease of G6PDH activity. This additional decrease is referred to as ‘inhibition,’ to distinguish it from the deactivation. Upon the addition of a sample containing the same analyte linked to G6PDH, some of the antibodies or receptors bound to the G6PDH-analyte conjugate now bind to the free analyte in the sample and release the G6PDH-analyte conjugate leading to an increase in G6PDH activity. This increase is referred to as ‘reversible inhibition.’ Once calibrated, as described herein, the analyte concentration in the sample is measured in terms of increased G6PDH enzyme activity. Thus, the assay is based on competition between the G6PDH-analyte conjugate and the free analyte in the sample for a fixed amount of specific antibody(ies) or receptor(s).

In the absence of analyte(s) in the sample, the specific antibody(ies) or receptors remain bound to the G6PDH-analyte conjugate causing no change in enzyme activity. On the other hand, when analyte(s) is(are) present in the sample, antibody(ies) or receptors would bind to the free analyte(s) in the sample and the enzymatic activity of the now unbound G6PDH-analyte conjugate is increased (‘reversible inhibition’). Thus, the activity of G6PDH depends upon the concentration of the analyte in the sample. The greater the analyte concentration in a sample, such as oral fluid, the greater the activity of G6PDH. Enzymatic activity is determined by measuring the formation of reduced nicotineamide adenine dinucleotide (NADH) at 340 nm. Thus, a change in absorption, measured in absorbent or milli-absorbant units, can be correlated to analyte concentration in a given sample.

In a preferred embodiment of the invention, the homogeneous enzyme immunoassay has a dynamic range of 0-100 ng/ml and produces an absorbance signal within the dynamic range from 0 to greater than 100 milli-absorbant units with a coefficient of variation of less than 10%.

In one embodiment of the invention, the homogeneous enzyme immunoassay has a dynamic range of 0-50 ng/ml. In another embodiment of the invention, the homogeneous enzyme immunoassay has a dynamic range of greater than 100 ng/ml.

To carry out the methods of this invention, the concentrations of the antibody(ies) or receptor(s) and G6PDH-analyte conjugate in the system are adjusted so that a desired % inhibition is achieved.

The extent of deactivation of the G6PDH due to conjugation with analyte(s) and the extent of inhibition of the G6PDH-analyte conjugate due to binding with antibodies (or receptors) is determined by conventional procedures as described herein, the EMIT literatures and U.S. Pat. No. 3,817,837.

In a preferred embodiment of the invention, the G6PDH is deactivated by from about 10% to about 85%, preferably from about 20% to about 85%, more preferably from about 40% to about 85%, and most preferably from about 30% to about 65%.

In another preferred embodiment of the invention, the deactivated enzyme-analyte conjugate is further inhibited by from about 20% to about 85%, preferably from about 30% to about 85%, and more preferably from about 40% to about 85%. In another embodiment, inhibition is from about 30% to about 65%.

The solvent for the homogeneous enzyme immunoassay is an aqueous medium. The aqueous medium may contain up to 40 weight percent, more usually less than about 20 weight percent, preferably less than 10 weight percent of other polar solvents, particularly oxygenated solvents of from 1-6, more preferable of from 1-4 carbon atoms, including alcohols, ethers and the like. Other useful solvents include, but are not limited to, DMF (dimethylformamide, N,N-dimethylformamide and DMS (dimehyl sulfide), and the like.

The pH for the assay will usually be in the range of between 4.0 and 11.0, more usually between 5.0 and 10.0, preferably in the range of between 6.0 and 9.0, more preferably between 7.0 and 8.5, and most preferably between 7.2 and 8.3. In one embodiment of the invention, the pH for the assay will be in the range of between 7.2 and 7.8.

Moderate temperatures are normally employed for carrying out the homogeneous enzyme immunoassay. Acceptable temperatures employed in the methods of this invention are temperatures, at which the enzyme-analyte conjugate, in particular the G6PDH-analyte conjugate, has enzymatic activity and thus, produces a detectable signal and at which the antibody or receptor can bind the analyte. Usually, temperatures for the assay will be in the range of about 4° C. to 50° C., more usually in the range of about 10° C. to 40° C., preferably in the range of 20° C. to 40° C., more preferably in the range of 30° C. to 40° C., most preferably at 37° C. It is well known in the art that enzymatic activities of isoforms, enzymes from different species, or mutated enzymes of a naturally occurring enzyme may be different at different temperatures. Thus, temperatures resulting in a desired high specific enzymatic activity may have to be determined empirically. Those methods are known in the art.

In carrying out the assay, the order of addition is not critical. The order of addition of the various reagents may very widely. In a preferred embodiment, the oral fluid sample is first combined with a reagent solution (referred to as R₁ in the Examples) comprising an antibody or receptor, substrate and co-factors for G6PDH. After incubation, the G6PDH-analyte conjugate (referred to as R₂ in the Examples). After another incubation, the signal generated is measured as described herein.

In one embodiment of the invention, the order of combining the reagents is as follows: (1) enzyme-analyte conjugate, (2) antibody or receptor reactive to the analyte, (3) substrate and co-enzyme for enzyme, and (4) oral fluid sample suspected of containing the analyte. Upon addition of the oral fluid sample containing an analyte, an increase in enzymatic activity (reversible inhibition) should be observed if the oral fluid sample contained an analyte as described herein.

In another preferred embodiment of the invention, the order of addition is as follows: (1) enzyme-analyte conjugate, (2) oral fluid sample suspected of containing the analyte, (3) antibody or receptor reactive to the analyte, and (4) substrate and co-enzyme for the enzyme. Upon addition of the substrate and co-enzyme the enzymatic activity is measured. The more analyte in the oral fluid sample, the more antibody or receptor will bind to the free analyte and not to the enzyme-analyte conjugate, thereby leading to a higher enzyme activity. If no analyte is present in the sample, then the antibody or receptor will bind exclusively to the enzyme-analyte conjugate and enzyme activity will be inhibited. Affecting the order of addition is whether an equilibrium mode or rate mode is employed.

Thus, one or more incubation steps may be involved in performing the homogeneous enzyme immunoassay. For example it will be desirable to incubate the enzyme-analyte conjugate with an antibody or receptor reactive to the analyte and to purify the enzyme-analyte conjugate with bound antibody or receptor before adding the oral fluid sample.

Whether to employ an incubation period and the length of the incubation period, will depend to a substantial degree on whether an equilibrium or rate mode is employed and the rate of binding of the antibody or receptor to the analyte. Usually, incubation steps will vary from about 5 seconds (secs) to 6 hours (hrs), more usually from about 30 secs to 1 hr.

### Measuring NADH—Autoanalyzer

The enzymatic activity of G6PDH can be measured by quantitative, semi-quantitative and qualitative methods. G6PDH enzymatic activity is determined by adding glucose-6-phosphate and NAD⁺ or NADP⁺ to the assay medium and detecting either the disappearance of one of these substrates or the appearance of NADH, NADPH, or D-glucono-δ-lactone-6-phosphate. Typically, the production of NADH or NADPH per unit time (usually in minutes) is measured using a spectrophotometer.

The time for measuring the signal will vary depending on whether a rate or equilibrium mode is used, the sensitivity required, the nature of the signal producing system and the like. For rate mode the times between readings will generally vary from about 5 seconds to 2 minutes, usually from about 30 seconds to 90 seconds, more usually from about 10 seconds to 60 seconds. For the equilibrium mode, after a steady state is achieved, a single reading may be sufficient or two readings over any convenient time interval may suffice.

Measuring the signal produced by the the methods of this invention can be applied easily to automated analyzers for laboratory, clinical, or high-throughput analysis. Examples of automated laboratory analyzers are COBAS INTEGRA and ROCHE/HITACHI series analyzers (Roche Diagnostics, Indianapolis, Ind.) and Olympus series (Texas). Generally, chemistry analyzers capable of maintaining a constant temperature, pipetting 30 μl to 70 μl of sample, mixing reagents, measuring enzyme rates at 340 nm wavelength, and timing the reaction accurately can be used to perform the method of the invention.

Other methods for measuring NADH or NADPH are also contemplated. For example, Babson and Babson (Clinical Chemistry 19(7):766-769, [1973]) described a method wherein the reduction of NAD is coupled to the reduction of a tetrazolium salt, 2-p-nitrophenyl-5-phenyl tetrazolium chloride (INT), with phenazine methosulfate serving as an intermediate electron carrier.

The signal producing system may also include G6PDH and a chromophoric substrate, where the chromophoric substrate is enzymatically converted to dyes which absorb light in the ultraviolet or visible region. Phosphors or fluorescers substrate are also contemplated by this invention.

Other detection methods will be apparent to those skilled in the art. By appropriate choice of components for producing a detectible signal, the detectible signal nay be observed visually or by means of various apparatus, i.e., detection means, such as spectrophotometers, fluorometers, scintillation counters, etc.

Various techniques and combinations of reagents may be employed to enhance the production of the detectible signal.

### Kits for Determining Analyte in Oral Fluid Sample

The invention also provides kits for conveniently testing the presence and accurately and reliably determining the amount of an analyte in an oral fluid sample suspected of containing an analyte. Kits of the invention may contain one or more of the following components as fully described herein: (a) an enzyme-analyte conjugate comprising glucose-6-phosphatase dehydrogenase (G6PDH) covalently linked to an analyte, (b) an antibody or receptor reactive to the analyte, (c) an enzyme substrate for G6PDH, (d) a co-enzyme for G6PDH, (e) a buffer, (f) calibrators or standards and the like, and (g) an instruction manual describing how to perform the homogenous enzyme immunoassay.

In one embodiment of the invention, reagents and compositions useful in the methods of the invention, are provided in a packaged combination. The reagents or compositions may be in the same or in separate containers depending on cross-reactivity and/or stability of the reagents or compositions. The reagents or compositions may be in liquid or in lyophilized form. Where reagents or compositions are provided as dry powders, i.e. usually lyophilized, excipients or buffers are included, so that upon dissolution, the reagent solutions will have the appropriate concentrations for performing the methods of this invention.

In one embodiment of the invention, the kit includes two or more different G6PDH-analyte conjugates. These two or more G6PDH-analyte substitutes can be used to determine the amount of two or more analytes in an oral fluid sample either subsequently or simultaneously as described in U.S. patent application Ser. No. 10/163,018 (Publication No. US-2003-0224373-A1), hereby incorporated in its entirety.

In one embodiment of the invention, a kit for blood testing of rehabilitated drug addicts or probational criminals is provided. This kit comprises two or more G6PDH-analyte conjugates wherein the conjugates comprise common drugs of abuse, such as THC/marijuana, morphine or heroin, PCP, amphetamines, methadone, methadone metabolite propoxyphene, and cocaine, etc.

In another embodiment of the invention, a kit for testing hospital patients is provided. This kit comprises two or more G6PDH-analyte conjugates wherein the conjugates comprise licit or illicit drugs as fully described herein. One kit for instance may comprise conjugates for commonly used illicit drugs for pre-employment drug-screening which typically include the so-called NIDA-5 (The National Institute on Drugs of Abuse) panel: opiate, cocaine, THC/marijuana, PCP, and amphetamines (include both amphetamine and methamphetamine).

Another embodiment of the invention provides a kit comprising two or more G6PDH-analyte conjugates wherein the conjugates comprise licit drugs that may commonly be taken in excess or whose presence need be ascertained in order to properly treat patients. Such a kit may include, for instance, conjugates comprising barbiturates, salicylate, tricyclic antidepressants such as imipramine, desipramine, amitriptyline, and nortriptyline, etc.

Another embodiment of the invention provides a kit for testing prospective employees. This kit comprises two or more G6PDH-analyte conjugates wherein the conjugates comprise alcohol, diuretics, cardiovascular drugs, and the like.

In one embodiment of the invention, a kit for testing exposure to industrial chemicals is provided. This kit comprises two or more G6PDH-analyte conjugates wherein the conjugates comprise common hazardous chemicals, or chemicals relevant to a particular site or occupation. Such kits may comprise conjugates directed to certain solvents, chemical intermediates, expected products, and the like. Similarly, kits used to monitor workers or others for exposure to pesticides may be prepared, with conjugates comprising the type of pesticides, or specific pesticides, in question.

In another embodiment of the invention, a kit for testing the presence of a chemical or biological warfare agent is provided. This kit comprises two or more G6PDH-analyte conjugates wherein the conjugates comprise a nerve agent (e.g., Sarin, Tabun, and Soman, etc.), mustard gas, Staphylococcus B Enterotoxin, Botulinum Toxin, Anthrax antigen(s), and smallpox antigen(s).

The invention is further illustrated by the following examples, which are only illustrative and are not intended to limit the definition of the invention in any way.

### EXAMPLES

### Example 1

### Calculation of Enzymatic Activity of the Enzyme-Analyte Conjugate

In order to measure accurately the analyte concentration in a sample suspected of containing an analyte, the signal (expressed as ΔA/min) generated between a negative calibrator, i.e., a calibrator with 0 ng/ml analyte and high calibrator, such as a calibrator with 50 ng/ml analyte by the G6PDH preferably should be at about 100 mA/min (rate mode). Thus, in a typical homogeneous enzyme immunoassay of this invention, G6PDH generates about 100 mA/min. The following equation states the relationship of signal intensity, enzyme activity, and reaction volume.
Enzyme Activity=ΔA×Vₜ/_{NADH}×V_{R2},
wherein ΔA is the signal generated by G6PDH (expressed in absorbent or milli-absorbant units); Vₜ is the total reaction volume in milliliter (ml) and includes volume of test sample, R₁ (volume of antibody or receptor, substrate, co-factor), and R₂ (volume of enzyme-analyte conjugate); [symbol] NADP is the extinction coefficient of NADH, corresponding to 6,220; and V_{R2} is the enzyme reagent volume in milliliter.

To meet the minimum volume requirement of currently available automated analyzers, such as the Hitachi 717, the total volume per immunoassay should be within 250 μl, including sample volume, enzyme reagent volume and antibody or receptor volume. In this example, using 20 μl of sample (such as oral fluid), 150 μl of antibody or receptor (R₁), and 75 μl of enzyme reagent (R₂), in order to generate 100 mA/min of signal, the required enzymatic activity would be 0.0525 units/ml. This is calculated as follows:
ΔA=0.1A(100 mA)
Vₜ₌0.02 ml+0.150 ml+0.075 ml=0.245 ml [symbol]
_{NADH}=6,220 molar extinction coefficient (=6.22 millimolar extinction coefficient)
Enzymatic activity=(0.1×0.245)/(6.22×0.075)=0.0525 units/ml

The calculated enzyme activity of 0.0525 units/ml is the effective (active) enzyme amount required to generate a 100 mA/min difference between the inhibited (negative analyte) and reversed (by high calibrator analyte) enzyme conjugate. In other words, 0.0525 units/ml of enzymatic activity would be required from negative calibrator (maximum inhibition) to high calibrator (50 ng/ml; reversible inhibition). This is a combination of native enzyme specific activity (i.e., before conjugation to analyte), enzymatic activity of the enzyme-analyte conjugate (deactivation), antibody or receptor inhibition, and reversible inhibition due to analyte in the test sample.

In order to meet the 0.0525 units/ml enzyme activity requirement and also in consideration of (1) the starting specific activity of the native G6PDH, (2) % of deactivation due to analyte conjugation, (3) % inhibition by antibody or receptor (taking into account antibody affinity for the analyte, Ka), (4) % reversible inhibition due to competition of antibody or receptor with free analyte in the sample (also known as modulation), the following conditions are recommended:
(a) A starting specific activity of the native G6PDH of at least 800 units/mg, preferably greater than 900 units/mg.
(b) Analyte-enzyme conjugate, which has an enzymatic activity of more than 400 units/mg of protein at 37° C.
(c) An antibody or receptor having an affinity to the analyte of Ka>5×10⁸M⁻¹.
(d) An inhibition of the enzyme-analyte conjugate due to the binding of the antibody or receptor by more than 60%.
(e) Oral fluid sample (saliva) appropriately diluted with oral fluid buffer.
(f) Appropriate volume of the oral fluid sample (saliva).

### Example 2

### G6PDH-PCP Conjugate

G6PDH with a starting specific activity of 860 units/mg was purchased from USB Biochemical. Nineteen (19) mg of the G6PDH was conjugated with PCP hapten leading to 45% of deactivation. After purification, 13 ml of enzyme-PCP conjugate (1.4 mg/ml) was isolated. The purified enzyme-PCP conjugate was further inhibited by up to 60% upon binding of antibody reactive to PCP. An enzyme reagent at 1 to 2,000 fold of dilution was formulated which contained 0.731 μg/ml of enzyme-PCP conjugate. In a desirable immunoassay, 20μ-45 μl of sample, 75 μl of enzyme-PCP conjugate, and 150 μl of antibody solution were used. The following calculation illustrate the importance of % deactivation, % inhibition, and sample volume.

| | | |
|---|---|---|
| 1. Enzyme concentration in the reagent: | 0.000731 | mg/ml |
| 2. Enzyme per assay (75 μl per assay): | 0.0000548 | mg/assay |
| 3. Normalize to 1.0 ml (from 0.245 ml | 0.000224 | mg/ml |
| assay volume): | | |
| 4. Enzyme activity after 45% deactivation: | 0.1058 | units/ml |
| 5. Needed enzyme to generate 100 mA: | 0.0525 | units/ml |
| 6. Sample 20 μl will have 30% reversible | 0.0370 | units/ml |
| inhibition: | | |
| 7. Signal generated by 0.0370 unit of enzyme: | 70.5 | mA |
| 8. Sample 45 μl wiil have 51% reversible | 0.0540 | units/ml |
| inhibition: | | |
| 9. Signal generated by 0.0540 unit of enzyme: | 102.8 | mA |

The following calculation is used:
1. = (19 mg/13 ml)/2,000 = 0.000731 mg/ml
2. = 0.000731 × 0.075/1 = 0.0000548 mg/assay
3. Normalize to 1 ml of volume from 0.245 ml (20 μl + 150 μl + 75 μl/assay)
4. = 0.000224 × 860 units/mg × (1-45%) = 0.1058 units/ml
5. See calculation herein (0.0525 unit/ml)
6. Experimental observation, related to antibody affinity.
7. = 100 mA × 0.0370/0.0525 = 70.5 mA
8. Experimental observation, related to antibody affinity.
9. = 100 mA × 0.0540/0.0525 = 102.8 mA

The results are illustrated Example 3 in the PCP oral fluid homogeneous enzyme immunoassay.

### Example 3

### G6PDH-Opiate Conjugate

G6PDH enzyme (4 mg, starting specific activity 860 units/ml) was conjugated with opiate hapten. 8.5 ml of G6PDH-opiate conjugate was purified. The conjugation resulted in 45% of deactivation of G6PDH. Opiate antibody binding to the G6PDH-opiate conjugate resulted in 52% of inhibition. The conjugate was formulated to the enzyme reagent at 1 to 450 dilution. By the same way of calculation as shown in Example 1, the following results are obtained:

| | | |
|---|---|---|
| 1. Enzyme concentration in the reagent: | 0.00106 | mg/ml |
| 2. Enzyme per assay (75 μl per assay): | 0.0000784 | mg/assay |
| 3. Normalize to 1.0 ml (from 0.245 ml | 0.000320 | mg/ml |
| assay volume): | | |
| 4. Enzyme activity after 45% deactivation: | 0.1514 | units/ml |
| 5. Needed enzyme to generate 100 mA: | 0.0525 | units/ml |
| 6. Sample 20 μl will have 15% reversible | 0.02270 | units/ml |
| inhibition: | | |
| 7. Signal generated by 0.0370 unit of enzyme: | 43.3 | mA |
| 8. Sample 45 μl will have 20% reversible | 0.0303 | units/ml |
| inhibition: | | |
| 9. Signal generated by 0.0540 unit of enzyme: | 57.7 | mA |

The lower reversibility of inhibition of #6 and #8 is due to the low affinity of the opiate antibody. The experimental results are illustrated in Examples 4 and 11.

### Example 4

### Importance of Sample Volume in Analyzing PCP and Opiate

G6PDH-PCP and G6PDH-opiate conjugates were prepared and analyzed as described herein. Keeping the total assay volume at 250 μl, PCP and opiate oral fluid samples of different volumes were analyzed: for PCP, 20 μl and 45 μl; for opiate, 20 μl and 50 μl.

| | PCP | | | | | |
|---|---|---|---|---|---|---|
| | 45 μl sample | | | 20 μl sample | | |
| Assay | (ng/mL) | Mean. | % CV | (ng/mL) | Mean. | % CV |
| Precision | 3 ng/mL | 2.4 | 6.35% | 3 ng/mL | 2.8 | 8.18% |
| | 5 ng/mL | 4.8 | 4.70% | 5 ng/mL | 4.9 | 6.90% |
| | 10 ng/mL | 11.2 | 5.36% | 10 ng/mL | 10.6 | 8.09% |
| Sensitivity | 1.6 ng/ml | | | 5 ng/ml | | |
| | Opiate | | | | | |
| | 50 μl sample | | | 20 μl sample | | |
| Assay | (ng/mL) | Mean. | % CV | (ng/mL) | Mean. | % CV |
| Precision | 10 ng/mL | 9.1 | 15.29% | 10 ng/mL | 9.9 | 27.8% |
| | 20 ng/mL | 18.3 | 8.12% | 20 ng/mL | 18.3 | 19.3% |
| | 30 ng/mL | 28.4 | 11.06% | 30 ng/mL | 29.7 | 12.2% |

| | | |
|---|---|---|
| Sensitivity | 3 ng/ml | 6 ng/ml |

By increasing the sample volume for the oral fluid sample, the sensitivity of detecting and measuring an analyte is increased and the % CV is decreased. Thus, for example, using 20 μl of samples with various PCP concentrations, 5 ng/ml PCP can be detected. However, increasing the sample volume to 45 μl allows detection of 1.6 ng/ml PCP, thus clearly increasing the sensitivity of the homogeneous enzyme immunoassay. Similarly, using 20 μl of samples with various opiate concentrations, 6 ng/ml opiate can be detected. However, increasing the sample volume to 50 μl, allows detection of 3 ng/ml opiate, thus, again, clearly increasing the sensitivity of the homogeneous enzyme immunoassay.

Typically, 21 samples were analyzed for each sample (Mean.) and the coefficient of variation is given (% CV).

### Example 5

### Preparation of Enzyme-Analyte Conjugate

Hapten Activation: A number of haptens (opiate, amphetamine, methamphetamine, benzoylecgonine, phencyclidine, methadone, methadone metabolite, MDMA) were purchased from commercial sources or obtained by custom synthesis contract services. All haptens were activated according to the procedure reported in U.S. Pat. No. 3,817,837 or U.S. patent application Ser. No. 10/163,018 (Publication No. US-2003-0224373-A1). Hapten, N-hydroxy succinimide and 1-(3-dimethylpropyl)-3-ethylcarbodiimide are dissolved in anhydrous DMF and the solution is stirred for 3 to 4 hours before conjugation.

Enzyme Solution: G6PDH enzyme in ammonium sulfate suspension was dialyzed against 50 mM Tris buffer, pH 8.1 and then adjusted to the final concentration at 3-10 mg/ml. Recombinant enzyme was dissolved in 50 mM of Tris buffer, pH 8.1 at concentration of 6 mg/ml.

Conjugation: Activated hapten is transferred into a proper size syringe and slowly added to the stirring enzyme solution via a syringe pump. The conjugation is carried out in a cold room and monitored by periodically measurements of the enzyme deactivation (% deactivation) and inhibition (% inhibition) by the analyte specific antibody. The conjugation is terminated at desirable % inhibition and the resulting crude conjugate is purified by a Sephdex G50 column with sodium azide as preservative. Benzoylecognine enzyme conjugate is carried out by directly adding the thioisocyanated hapten. The conjugate is monitored and worked up by the same method.

The following G6PDH-analyte conjugates are suitable for use in homogeneous enzyme immunoassays analyzing oral fluid samples suspected of containing an analyte:

| Conjugate | % Deactivation | % Inhibition |
|---|---|---|
| Amphetamine | 40% | 70% |
| Benzoylecgonine | 55% | 60% |
| Ecstasy (MDMA) | 55% | 65% |
| Opiate | 50% | 65% |
| Methamphetamine | 40% | 78% |
| Methadone | 55% | 60% |
| EDDP | 45% | 70% |
| PCP | 45% | 65% |

### Example 6

### Calibrators and Assay Reagents

Calibrators and controls: The following table illustrates calibrators and controls for each analyte. Both calibrators and controls are prepared by spiking analytes into negative oral fluid buffer. Each analyte concentration is designed to follow the SAMHSA's guidelines.

| Analyte | Cont. I | Cutoff cal. | Cont. II | High cal. |
|---|---|---|---|---|
| Amphetamine | 15 ng/ml | 25 ng/ml | 35 ng/ml | 50 ng/ml |
| Cocaine | 5 ng/ml | 10 ng/ml | 20 ng/ml | 50 ng/ml |
| Methamphetamine | 15 ng/ml | 25 ng/ml | 35 ng/ml | 50 ng/ml |
| MDMA | 15 ng/ml | 25 ng/ml | 35 ng/ml | 50 ng/ml |
| Methadone | 10 ng/ml | 20 ng/ml | 30 ng/ml | 50 ng/ml |
| EDDP | 10 ng/ml | 20 ng/ml | 30 ng/ml | 50 ng/ml |
| Opiate | 10 ng/ml | 20 ng/ml | 30 ng/ml | 50 ng/ml |
| Phencyclidine | 3 ng/ml | 5 ng/ml | 10 ng/ml | 15 ng/ml |

Cont., control;
cal., calibrator.
Typically, calibrators are used to calibrate the reagents and controls are used to validate the reagents.

Antibody buffer: 20 mM Tris buffer containing 40 mM G6P, 35 mM β-nicotinamide adenine dinucleotide (NAD), 0.5% sodium chloride, 0.09% sodium azide, 0.1% BSA, pH 5.4.

Antibody reagent: The monoclonal antibodies was diluted into the antibody buffer. Each antibody inhibited the enzyme activity approximately 50%-60%.

Enzyme buffer: 50 mM Tris buffer containing 0.9% sodium chloride, 0.09% sodium azide, 0.1% BSA, pH 8.2.

Enzyme reagent: The hapten-labeled enzyme conjugate was diluted into the enzyme buffer at a concentration which would result in a maximum rate of about 200 mA-550 mA per minute as measured at 37° C. according to the assay protocol described herein.

### Example 7

### Assay Protocol

One hundred and fifty microliters (150 μl) of antibody reagent was incubated with 40 μl to 50 μl of calibrator or specimen for 300 seconds at 37° C., followed by addition of 75 μl of the enzyme reagent. The solution was incubated at 37° C. for 20 seconds before the first optical absorbance measurement was taken at 340 nm. The second optical absorbance measurement was taken at 60 seconds after the first measurement.

The optical absorbance difference between the first and the second measurements was divided by the interval time and recorded as mA/min (rate mode). A maximum enzyme rate was measured by substituting the antibody reagent with the antibody buffer.

### Example 7

### Calibration and Determination of Amphetamines in Oral Fluid Homogeneous Enzyme Immunoassay

The following data were obtained using an homogeneous enzyme immunoassay for detecting and measuring the amount of amphetamine in an oral fluid sample. G6PDH-amphetamine conjugates, antibodies, and oral fluid calibrators were prepared and used as described herein.

| Cal./Contl, ng/ml | Rate, mA/min |
|---|---|
| 0 | 264 |
| 15 | 305 |
| 25 | 325 |
| 35 | 340 |
| 50 | 360 |

Cal./Contl., calibration/control.
The data obtained were used to generate the calibration standard curve shown in FIG. 1.

### Example 8

### Calibration and Determination of Phencyclidine (PCP) in Oral Fluid Homogeneous Enzyme Immunoassay

The following data were obtained using an homogeneous enzyme immunoassay for detecting and measuring the amount of phencyclidine (PCP) in an oral fluid sample. G6PDH-phencyclidine conjugates, antibodies, and oral fluid calibrators were prepared and used as described herein.

| | | |
|---|---|---|
| Sample: | 45 μl | 20 μl |
| Ab: | 150 μl | 150 μl |
| Enz: | 75 μl | 75 μl |
| Total volume | 265 μl | 245 μl |

| Cal./Contl. (ng/ml) | Rate, mA/min | Rate, mA/min |
|---|---|---|
| 0 | 175.8 | 179.0 |
| 3 | 194.6 | 188.1 |
| 5 | 208.8 | 195.3 |
| 10 | 239.3 | 209.0 |
| 25 | 284.3 | 254.0 |
| Total separation | 108.5 | 75.0 |

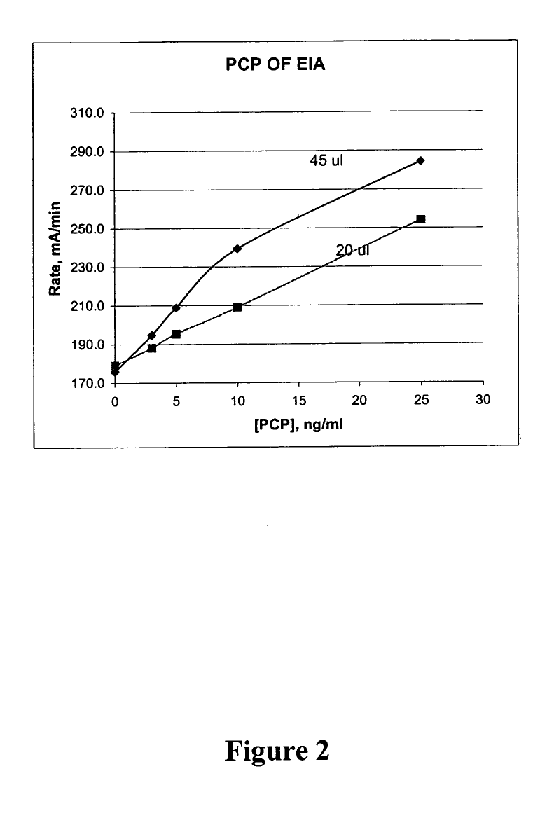

Cal./Contl., calibration/control; Ab, antibody; Enz, enzyme-phencyclidine conjugate. Total separation refers to the rate difference between the negative calibrator (0 ng/ml analyte) and the high calibrator. The data obtained were used to generate the calibration standard curve, shown in FIG. 2.

### Example 9

### Phencyclidine (PCP) Assay Precision Using Oral Fluid Homogeneous Enzyme Immunoassay and Different PCP Sample Volumes

The following data were obtained using an homogeneous enzyme immunoassay for detecting and measuring the amount of phencyclidine (PCP) in an oral fluid sample. G6PDH-phencyclidine conjugates, antibodies, and oral fluid calibrators were prepared and used as described herein. Two different volumes of oral fluid sample, 20 μl and 45 μl were analyzed in a total of 12 replicates of three different phencyclidine concentrations, 3 ng/ml, 5 ng/ml, and 10 ng/ml.

| 45 μl sample volume | | | | 20 μl sample volume | | | |
|---|---|---|---|---|---|---|---|
| Replicate | 3 ng/ml | 5 ng/ml | 10 ng/ml | Replicate | 3 ng/ml | 5 ng/ml | 10 ng/ml |

| | | | | | | | |
|---|---|---|---|---|---|---|---|
| 1 | 2.3 | 5.3 | 12.7 | 1 | 2.7 | 4.8 | 10.7 |
| 2 | 2.1 | 5.1 | 11.2 | 2 | 2.6 | 4.7 | 9.3 |
| 3 | 2.2 | 4.8 | 10.7 | 3 | 2.9 | 5.3 | 11.3 |
| 4 | 2.3 | 4.8 | 10.9 | 4 | 3.0 | 4.7 | 11.7 |
| 5 | 2.5 | 4.9 | 11.1 | 5 | 2.6 | 4.6 | 9.8 |
| 6 | 2.3 | 4.8 | 10.6 | 6 | 2.8 | 4.4 | 10.1 |
| 7 | 2.3 | 4.7 | 11.2 | 7 | 2.6 | 5.1 | 10.3 |
| 8 | 2.6 | 4.7 | 11.3 | 8 | 2.6 | 4.7 | 10.1 |
| 9 | 2.5 | 4.6 | 11.4 | 9 | 2.6 | 4.9 | 9.8 |
| 10 | 2.5 | 4.5 | 10.5 | 10 | 2.9 | 5.5 | 11.6 |
| 11 | 2.6 | 4.9 | 10.9 | 11 | 3.1 | 5.0 | 11.5 |
| 12 | 2.5 | 5.1 | 11.7 | 12 | 3.2 | 5.2 | 11.1 |
| Avg. | 2.4 | 4.8 | 11.2 | Avg. | 2.8 | 4.9 | 10.6 |
| Std. | 0.15 | 0.23 | 0.60 | Std. | 0.23 | 0.34 | 0.86 |
| % CV | 6.35% | 4.70% | 5.36% | % CV | 8.18% | 6.90% | 8.09% |

Avg., average;
Std., standard deviation;
% CV, percent coefficient of variation.

### Example 10

### Calibration and Determination of Opiate in Oral Fluid Homogeneous Enzyme Immunoassay

The following data were obtained using an homogeneous enzyme immunoassay for detecting and measuring the amount of opiate in an oral fluid sample. G6PDH-opiate conjugates, antibodies, and oral fluid calibrators were prepared and used as described herein.

| | | |
|---|---|---|
| Sample: | 50 μl | 20 μl |

| Cal./Contl. (ng/ml) | Rate, mA/min | Rate, mA/min |
|---|---|---|
| 0 | 270 | 332 |
| 10 | 287 | 342 |
| 20 | 306 | 349 |
| 30 | 319 | 359 |
| 50 | 330 | 376 |
| Total separation | 60 | 44 |

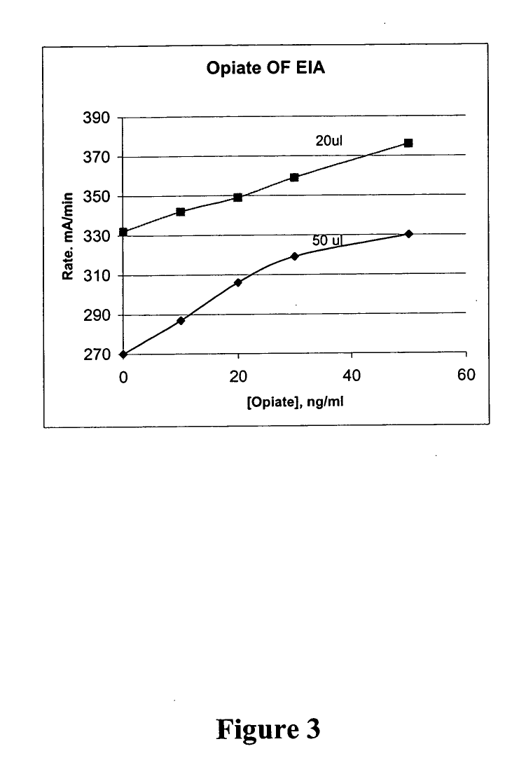

Cal./Contl., calibration/control.
Total separation refers to the rate difference between the negative calibrator (0 ng/ml analyte) and the high calibrator.
The data obtained were used to generate the calibration standard curve, shown in FIG. 3.

### Example 11

### Opiate Assay Precision Using Oral Fluid Homogeneous Enzyme Immunoassay and Different Opiate Sample Volumes

The following data were obtained using an homogeneous enzyme immunoassay for detecting and measuring the amount of opiate in an oral fluid sample. G6PDH-opiate conjugates, antibodies, and oral fluid calibrators were prepared and used as described herein. Two different volumes of oral fluid sample, 20 μl and 50 μl were analyzed in a total of 21 replicates of three different opiate concentrations, 10 ng/ml, 20 ng/ml, and 30 ng/ml.

| For 50 μl sample volume | | | | For 20 μl sample volume | | | |
|---|---|---|---|---|---|---|---|
| Replicate | 10 ng/ml | 20 ng/ml | 30 ng/ml | Replicate | 10 ng/ml | 20 ng/ml | 30 ng/ml |
| 1 | 10.8 | 19.5 | 29.3 | 1 | 12.8 | 22.5 | 33.2 |
| 2 | 9.5 | 15.7 | 26.5 | 2 | 10.2 | 14.3 | 29.6 |
| 3 | 8.0 | 18.1 | 27.0 | 3 | 6.5 | 14.5 | 26.7 |
| 4 | 7.7 | 17.3 | 28.6 | 4 | 9.6 | 21.7 | 27.5 |
| 5 | 6.8 | 16.7 | 25.2 | 5 | 6.4 | 14.8 | 36.5 |
| 6 | 6.7 | 19.4 | 28.6 | 6 | 7.6 | 18.6 | 29.7 |
| 7 | 9.1 | 18.1 | 22.3 | 7 | 9.5 | 22.9 | 24.3 |
| 8 | 8.4 | 16.2 | 26.4 | 8 | 7.1 | 13.6 | 28.9 |
| 9 | 8.4 | 21.0 | 28.4 | 9 | 6.5 | 24.7 | 33.2 |
| 10 | 10.8 | 17.2 | 32.1 | 10 | 12.3 | 15.2 | 31.5 |
| 11 | 10.7 | 20.1 | 31.1 | 11 | 11.5 | 22.1 | 32.6 |
| 12 | 10.6 | 20.6 | 32.7 | 12 | 15.3 | 21.5 | 26.8 |
| 13 | 10.0 | 18.7 | 32.9 | 13 | 11.2 | 15.8 | 31.7 |
| 14 | 8.4 | 17.9 | 22.4 | 14 | 8.9 | 18.6 | 21.8 |
| 15 | 8.9 | 16.4 | 29.6 | 15 | 15.6 | 13.6 | 33.2 |
| 16 | 8.0 | 18.6 | 26.2 | 16 | 7.6 | 18.9 | 33.6 |
| 17 | 9.3 | 17.7 | 31.4 | 17 | 8.7 | 16.4 | 30.2 |
| 18 | 8.1 | 18.1 | 25.2 | 18 | 9.8 | 19.2 | 25.8 |
| 19 | 8.5 | 18.4 | 28.6 | 19 | 7.8 | 16.7 | 25.6 |
| 20 | 11.0 | 20.4 | 30.3 | 20 | 10.4 | 22.4 | 29.8 |
| 21 | 11.3 | 17.7 | 32.3 | 21 | 13.2 | 15.8 | 31.7 |
| Avg. | 9.1 | 18.3 | 28.4 | Avg. | 9.9 | 18.3 | 29.7 |
| Std. | 1.4 | 1.5 | 3.1 | Std. | 2.8 | 3.5 | 3.6 |
| % CV | 15.3% | 8.1% | 11.1% | % CV | 27.8% | 19.3% | 12.2% |

Avg., average;
Std., standard deviation;
% CV, percent coefficient of variation.

### Example 12

### Calibration and Determination of Cocaine Metabolite in Oral Fluid Homogeneous Enzyme Immunoassay

The following data were obtained using an homogeneous enzyme immunoassay for detecting and measuring the amount of cocaine metabolite (benzo-ecgonine) in an oral fluid sample. G6PDH-cocaine metabolite conjugates, antibodies, and oral fluid calibrators were prepared and used as described herein.

| Cal./contl., ng/ml | Rate, mA/min |
|---|---|
| 0 | 226.9 |
| 5.0 | 252.7 |
| 10.0 | 266.0 |
| 20.0 | 282.2 |
| 50.0 | 311.7 |

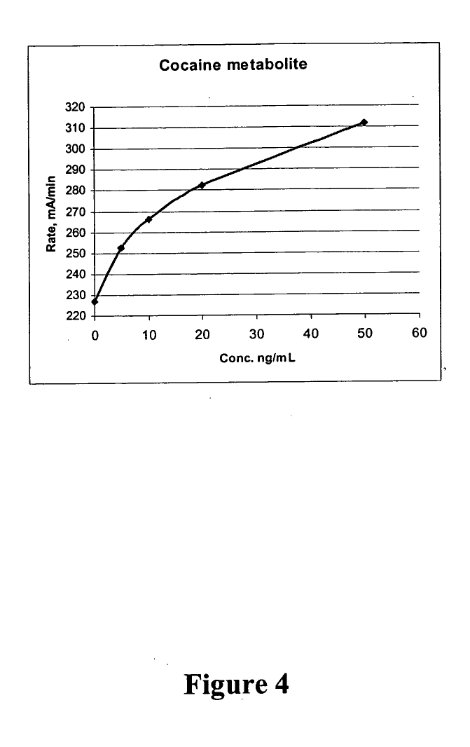

Cal./Contl., calibration/control.
The data obtained were used to generate the calibration standard curve, shown in FIG. 4.

### Example 13

### Calibration and Determination of Ecstasy (MDMA) in Oral Fluid Homogeneous Enzyme Immunoassay

The following data were obtained using an homogeneous enzyme immunoassay for detecting and measuring the amount of Ecstacy (MDMA) in an oral fluid sample. G6PDH-Ecstacy (MDMA) conjugates, antibodies, and oral fluid calibrators were prepared and used as described herein.

| Cal/contl., ng/ml | Rate, mA/min |
|---|---|
| 0 | 232 |
| 15 | 263 |
| 25 | 288 |
| 35 | 322 |
| 50 | 368 |

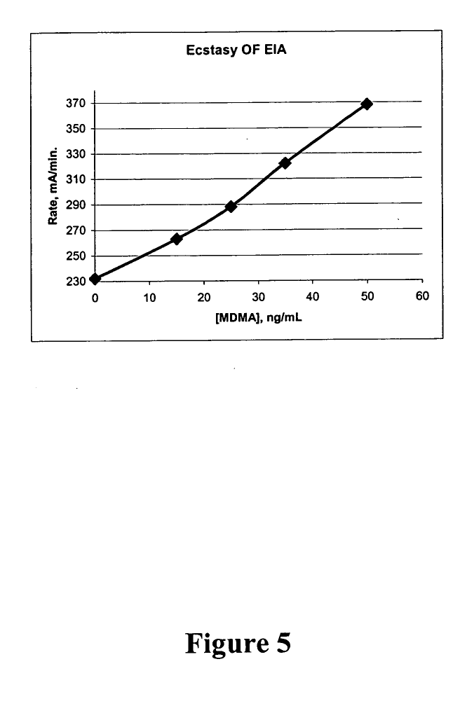

Cal/contl., calibration/control. The data obtained were used to generate the calibration standard curve, shown in FIG. 5.

### Example 14

### Calibration and Determination of Methadone Metabolite (EDDP) in Oral Fluid Homogeneous Enzyme Immunoassay

The following data were obtained using an homogeneous enzyme immunoassay for detecting and measuring the amount of methadone metabolite (EDDP) in an oral fluid sample. G6PDH-methadone metabolite (EDDP) conjugates, antibodies, and oral fluid calibrators were prepared and used as described herein.

| Cal/contl., ng/ml | Rate, mA/min |
|---|---|
| 0 | 216 |
| 10 | 251 |
| 20 | 300 |
| 30 | 347 |
| 50 | 415 |

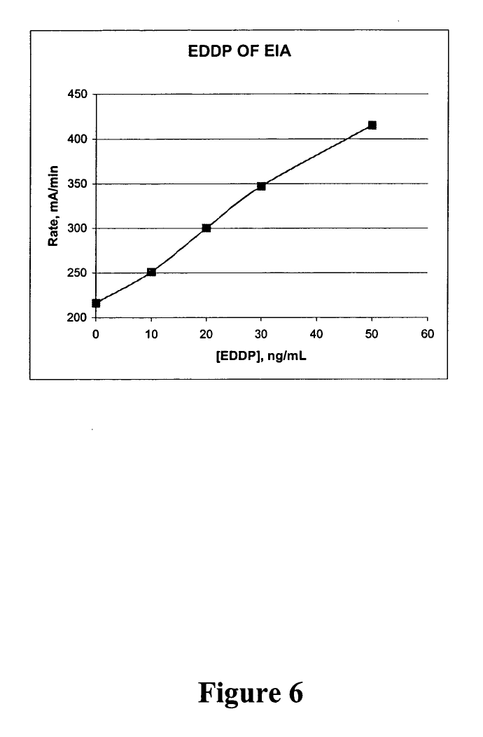

Cal./Contl., calibration/control. The data obtained were used to generate the calibration standard curve, shown in FIG. 6.

What is claimed is:

1. A homogeneous enzyme immunoassay system for determining the amount of an analyte in an oral fluid sample where a homogenous enzyme immunoassay has a dynamic range of 0-100 ng/ml and produces an absorbance signal within the dynamic range from 0 to greater than 100 milli-absorbant units with a coefficient of variation of less than 10%, the system comprising an aqueous medium comprising:
(a) an enzyme-analyte conjugate comprising glucose-6-phosphate dehydrogenase (G6PDH) covalently linked to an analyte;
(b) an antibody reactive to the analyte;
(c) an oral fluid sample suspected of containing the analyte;
(d) an enzyme substrate for G6PDH; and
(e) a co-enzyme for G6PDH; and further provided that:
(i) the G6PDH has a starting specific activity of at least 800 units/mg and the enzyme-analyte conjugate is deactivated from about 30% to about 65% due to covalent linkage of the G6PDH to the analyte; and
(ii) wherein the deactivated enzyme-analyte conjugate is further inhibited from about 40% to about 85% due to binding of the antibody to the analyte of the enzyme-analyte conjugate.

2. A homogeneous enzyme immunoassay system according to claim 1 wherein the oral fluid sample is buffered to a pH range from between 7.2 and 8.3.

3. A homogeneous enzyme immunoassay system according to claim 1 wherein the oral fluid sample is filtered or centrifuged.

4. A homogeneous enzyme immunoassay system according to claim 1 wherein the analyte is selected from the group consisting of licit drugs, illicit drugs and analogs, derivatives and metabolites thereof.

5. A homogeneous enzyme immunoassay system according to claim 1 wherein the analyte is selected from the group consisting of opium, opioid analgesics, amphetamines, cocaine, methadone, methadone metabolite, MDMA, PCP, propoxyphene, benzodiazepines, barbiturates, THC, alcohol and analogs, metabolites, and derivatives thereof.

6. A homogeneous enzyme immunoassay system according to claim 1 wherein the G6PDH is obtained from a natural source.

7. A homogeneous enzyme immunoassay system according to claim 1 wherein the G6PDH is a recombinant enzyme.

8. A homogeneous enzyme immunoassay system according to claim 2 wherein the oral fluid sample is between about 20 μl and about 50 μl in volume.

9. A homogeneous enzyme immunoassay system according to claim 3 wherein the oral fluid sample is between about 20 μl and about 50 μl in volume.

10. A method for determining the amount of an analyte in an oral fluid sample using a homogeneous enzyme immunoassay where the immunoassay has a dynamic range of 0-100 ng/ml and produces an absorbance signal within the dynamic range from 0 to greater than 100 milli-absorbant units with a coefficient of variation of less than 10%, the method comprising the steps of:
(I) combining in an aqueous medium:
(a) an enzyme-analyte conjugate comprising glucose-6-phosphate dehydrogenase (G6PDH) covalently linked to an analyte;
(b) an antibody reactive to the analyte;
(c) an oral fluid sample suspected of containing the analyte;
(d) an enzyme substrate for G6PDH; and
(e) a co-enzyme for G6PDH; and
(II) detecting a change in enzymatic activity of the enzyme-analyte conjugate due to competitive binding of the antibody bound to the analyte of the enzyme-analyte conjugate and the analyte in the oral fluid sample; and
further provided that:
(i) the G6PDH has a starting specific activity of at least 800 units/mg and the enzyme-analyte conjugate is deactivated from about 30% to about 65% due to covalent linkage of the G6PDH to the analyte;
(ii) wherein the deactivated enzyme-analyte conjugate is further inhibited from about 40% to about 85% due to binding of the antibody to the analyte of the enzyme-analyte conjugate; and
(iii) wherein the change in enzymatic activity is related to the amount of the analyte in the oral fluid sample.

11. A method according to claim 10 wherein the oral fluid sample is buffered to a pH range from between 7.2 and 8.3.

12. A method according to claim 10 wherein the oral fluid sample is filtered or centrifuged.

13. A method according to claim 10 wherein the analyte is selected from the group consisting of licit drugs, illicit drugs and analogs, derivatives and metabolites thereof.

14. A method according to claim 10 wherein the analyte is selected from the group consisting of opium, opioid analgesics, amphetamines, cocaine, methadone, methadone metabolite, MDMA, PCP, propoxyphene, benzodiazepines, barbiturates, THC, alcohol and analogs, metabolites, and derivatives thereof.

15. A method according to claim 10 wherein the G6PDH is obtained from a natural source.

16. A method according to claim 10 wherein the G6PDH is a recombinant enzyme.

17. A method according to claim 11 wherein the oral fluid sample is between about 20 μl and about 50 μl in volume.

18. A method according to claim 12 wherein the oral fluid sample is between about 20 μl and about 50 μl in volume.

19. A kit for use in an assay for determining the amount of an analyte in an oral fluid sample suspected of containing an analyte, the kit comprising, in a packaged combination the following reagent compositions:
(a) an enzyme-analyte conjugate comprising glucose-6-phosphate dehydrogenase (G6PDH) covalently linked to an analyte;
(b) an antibody reactive to the analyte;
(c) an enzyme substrate for G6PDH; and
(d) a co-enzyme for G6PDH.

20. A kit according to claim 19, further comprising an oral fluid calibrator.
